(19) United States
(12) Patent Application Publication
(10) Pub. No.: US 2003/0017149 A1
(43) Pub. Date: Jan. 23, 2003

(54) Single chain monoclonal antibody fusion reagents that regulate transcription in vivo

(76) Inventors: James P. Hoeffler, Carlsbad, CA (US); Marijane Russell, San Diego, CA (US)

Correspondence Address: Stephen A. Bent, FOLEY & LARDNER, Suite 500, 3000 K Street, N.W., Washington, DC, 20007-5109, US

(21) Appl. No.: 09/939,769

(22) Filed: Aug. 28, 2001

Related U.S. Application Data
(63) Continuation of application No. 08/728,890, filed on Oct. 10, 1996, now abandoned.

(51) Int. Cl. C12Q 1/68; G01N 33/53; G01N 33/567; C12P 21/06; A61K 39/395

(52) U.S. Cl. 424/130.1; 435/6; 435/7.2; 435/7.1; 435/69.1

(57) ABSTRACT

A method of screening a DNA construct library for a single chain monoclonal antibody s fusion reagent capable of binding a transcriptional associated biomolecule in vivo is described. Single chain monoclonal antibody fusion reagents capable of binding transcriptional associated biomolecules in vivo are provided. Single chain monoclonal antibody fusion reagents which are capable of regulating transcription in zivo are also provided. Therapeutic methods for regulating the transcription of a gene in vivo are also described. A method is further provided for screening a plurality of compounds for specific binding affinity with a single chain monoclonal antibody fusion reagent. A method is also described for diagnosing a physiological disorder manifested by an abnormal level of a transcription associated biomolecule. A DNA construct (pVP16Zeo) as well as primers for the construction and screening of single chain monoclonal antibody fusion reagent libraries to facilitate the isolation and production of single chain monoclonal antibody fusion reagents in yeast and E.coli are also provided. A kit for screening a DNA construct library for a single chain monoclonal antibody fusion reagent capable of binding a transcriptional associated biomolecule in vivo is also provided.

The present invention provides a method for screening DNA construct libraries for those which encode single-chain fragments of immunoglobulin variable domains (sFvs) having specificity for desired antigens in vivo using the activity of a transcriptional activator. More specifically, the present invention is directed to a method of screening for single-chain fragments of immunoglobulin variable domains capable of targeting transcription associated biomolecules in vivo. The present invention is also directed to monoclonal antibody fusion reagents that regulate transcription in vivo.

The invention described herein was supported in part by National Institutes of Health grant NIDDK R43DK51418.

### BACKGROUND

Antibody fragments binding with high affinity to their target can be obtained from hybiidomas or directly from antibody libraries on filamentous phage. Recombinant antibody fragments such as Fab, Fv, and sFv fragments can be efficiently expressed in bacteria and on the surface of filamentous phage and can be readily isolated. Neri, D., et al., Engineering Recombinant Antibodies for Immunotherapy, Cell Biophysics, 27:47 (1995); Grifiths, A. D., et al., EMBO J., 13:3245 (1994). Typically, recombinant antibodies are generated and expressed in bacteria by cloning repertoires of rearranged heavy and light chain V-genes into filamentous bacteriophage and selected for specificity from the phage library by panning with antigen. See e.g. Vaughan, T. j., et al., Nature Biotech., 14:309 (1996); De Kruif, J., et al., J. Mol. Biol., 248:97 (1995); Marks, J. D., et al., J. Mol. Biol., 222:581 (1991). The current method of choice to screen sFv libraries utilizes a bacterial phage system which displays the sFv's on the surface of the gene III protein of M13 phage. Hogenboom, H. R., et al., Nucleic Acids Res., 19:4133 (1991). The phage library is mixed with plates or columns coated with the antigen of interest and washed extensively to eliminate unbound or weakly bound phage. Phage are relatively resistant to acidic treatments needed to disrupt the sFv/Ag and can be eluted with acid and put through multiple rounds of selection to enhance specificity and affinity of the sFv selected. Alternatively, well established useful hybridomas can be used as sources for V_{H} and V_{L} for the production of recombinant antibodies using commercially available kits and protocols (e.g., Recombinant Phage Antibody System™, Pharmacia).

U.S. Pat. No. 5,427,908, issued Jun. 27, 1995, for example, provides recombinant library screening methods wherein nucleotide sequences which encode monoclonal antibodies of interest are isolated from DNA libraries using bacteriophage to link the antibody fragment to the sequence which encodes it. DNA libraries are prepared from cells encoding the antibody of interest and inserted into or adjacent to a coat protein of a bacteriophage vector, or into a sequence encoding a protein which may be linked by means of a ligand to a phage coat protein. By employing affinity purification techniques the phage particles containing sequences encoding the desired protein may be selected and the desired nucleotide sequences obtained.

Antibody selection/screening systems currently available continue to be hampered by the inherent lack of the ability to accurately predict immunorecognition properties in vivo.

Insights into the mechanisms underlying the regulation of gene expression have come about from studies of the structure and functions of eukaryotic transcription factors and the signaling pathways that regulate their activities. Cells respond to environmental changes by sensing substances known as ligands and hormones. Signal transduction involves binding of a hormone or ligand to a specific cell surface receptor which initiates a signaling cascade within the cell resulting in the activation of multiple specific protein kinases and/or phosphatases involved in cell growth which in turn influence the activity of specific transcriptional regulatory proteins. Pelech, S., et al., Biochem. Cell Biol., 68:1297 (1990); Hunter, T., Karin, M., Cell, 70:375 (1992). These signaling pathways converge ultimately at the level of the nucleus to influence specific patterns of gene expression that regulate growth. Hormonal activation of signal transduction pathways links extracellular signals to intracellular signals commonly referred to as second messengers which eventually influence transcriptional responses through transcription associated biomolecules resulting in the activation of many cellular genes. Malarkey, K., et al., Biochem J., 309:361 (1995).

The regulation of transcription in eukaryotes relies upon the in situ nature of DNA packaging and the histone proteins in several essential ways. Certain promoters make use of the staged assembly of chromatin in vivo and a rapid and tight association of trans-acting factors with promoter elements to remain constitutively active. Moreover, nucleosome folding of DNA by the histones can facilitate the activation of genes by bringing widely separated regulatory elements into juxtaposition. Thus, histones provide the necessary infrastructure for the correct and efficient operation of the transcriptional machinery; however, their exact contributions to the transcriptional regulation of an individual gene may depend on the spatial distribution of regulatory elements, the transcription factors involved, and the three-dimensional folding of DNA that they direct. Wolfe, A. P., Cell, 77 (1):13 (1994).

Attempts to study functional transcription corellates in vivo have relied predominantly on transient or stable expression of regulatory proteins and transcription factors. These approaches have suffered from the obvious shortcomings of overexpressing effectors that under normal conditions are stringently regulated. In addition to the common shortcomings of overexpressing the transcriptional effectors and risking non-physiologically relevant binding and activation from promoters due to straight competition at less optimal binding sequence, the overexpression of regulatory proteins and transcription factors generally has the disadvantage of ubiquitously expressed endogenous proteins that all bind the same consensus motif in vitro. This makes interpretation of these types of experiments almost impossible. Many of the studies to characterize eukaryotic transcription factors have been done in in vitro model systems which measure transcription factor intermolecular association with biomolecules and nucleic acids. Interpretation of the results of these studies has also been tempered by the obvious limitations of the in vitro systems.

The need therefore clearly exists for a novel assay system in which the functions of individual members of transcription factor families can be assessed under physiologically relevant conditions in vivo. More particularly a need exists for a method of screening for and isolating single-chain fragments of immunoglobulin variable domains capable of targeting characteristic transcription factors and related biomolecules in vivo.

### SUMMARY OF THE INVENTION

The present invention is directed to a method of screening a DNA construct library for a single chain monoclonal antibody fusion reagent capable of binding a transcriptional associated biomolecule in vivo.

A method is also provided for screening a DNA construct library for a construct which encodes a single chain monoclonal antibody fusion reagent that regulates transcription in vivo.

A method is also provided for screening a DNA construct library for a construct which encodes a single chain monoclonal antibody fusion reagent that regulates transcription in vivo comprising an intracellular targeting signal peptide (ITSP).

Single chain monoclonal antibody fusion reagents capable of binding transcriptional associated biomolecules and regulating transcription in vivo are also provided.

Preferred embodiments of the single chain fusion reagents have the general structures:

*NH₂—V_{H}—linker—V_{L}—transcriptional activator—COOH* (I)

*NH₂—ITSP—V_{H}—linker—V_{L}—transcriptional activator—COOH* (II)

*NH₂—ITSP—V_{H}—linker—V_{L}—ITSP—transcriptional activator—COOH* (III)

*NH₂—V_{H}—linker—V_{L}—COOH* (IV)

*NH₂—ITSP—V_{H} linker—V_{L}—COOH* (V)

*NH₂—ITSP—V_{H}—linker—V_{L}—ITSP—COOH* (VI)

The V_{H} and V_{L} regions of the single chain fusion reagents of the present invention may be reversed, i.e. V_{H}—linker—V_{L} or V_{L}—linker—V_{H}.

Single chain fusion reagents of the present invention may comprise a transcriptional repressor (TR) or a repressor interacting domain (RID) instead of a transcriptional activator.

An object of the present invention is to provide a method which can be used in the design of fusion reagents to be used therapeutically.

Another object of the invention is to provide a therapeutic method for regulating the transcription of a gene in vivo.

Another object of the invention is to provide a therapeutic method for regulating the function of a transcriptional associated biomolecule in vivo.

A still further object of the invention is to provide a method for diagnosing a physiological disorder manifested by an abnormal level of a transcription associated biomolecule.

A still further object of the invention is to provide a method of screening a plurality of compounds for specific binding affinity with a single chain monoclonal antibody fusion reagent.

A DNA construct and primers for the construction and screening of single chain monoclonal antibody fusion reagent libraries to facilitate the isolation and production of single chain monoclonal antibody fusion reagents in yeast and E.coli is also provided.

A kit for screening a DNA construct library for a single chain monoclonal antibody fusion reagent capable of binding a transcriptional associated biomolecule in vivo is also provided.

For a better understanding of the present invention, reference is made to the following description, taken together with the accompanying figures, and the scope of which is pointed out in the appended claims.

### BRIEF DESCRIPTION OF THE FIGURES

FIG. 1 Shows an example single chain monoclonal antibody fusion reagent that regulates transcription comprised of a nuclear localization signal, two immunoglobulin variable domains connected by a linker, fused to the example transcriptional activator, VP16. Also shown is an example LexA-DBD/CREBPBOX antigen (bait) fusion bound to the UAS of a reporter gene, and the transcriptional activation of the reporter gene via the example single chain monoclonal antibody fusion reagent that regulates transcription; also shown is an example of transcriptional regulation in vivo wherein the single chain monoclonal antibody fusion reagent that regulates transcription complexes with endogenous CREB-bound CRE and activates the transcription of native genes.

FIG. 2 Shows schematic representations of example intracellular targeting sequences for use with single chain monoclonal antibody reagents. Targeting vectors direct expression of sFvs to either the cytoplasm, nucleus, endoplasmic reticulum, or the mitochondria.

FIG. 3 Shows the pBTM116 yeast expression plasmid as an example vector used to construct antigen (X) “bait” strain fusions to screen the antibody fusion reagent library.

FIG. 4 Shows pVP16*, an example yeast expression plasmid vector used to express a library of human single chain immunoglobulin variable regions as single chain monoclonal antibody fusion reagents.

FIG. 5 Shows pVP16Zeo, an example of a yeast expression plasmid vector with a dual selectable marker, zeocin, used to express a library of human single chain immunoglobulin variable regions as single chain monoclonal antibody fusion reagents.

FIG. 6 Shows a schematic representation of an ATF-2FL transcription factor for use in example antigen (bait) fusion constructs. Also shown is a schematic representation of a CREB transcription factor for use in example antigen (bait) fusion constructs.

FIG. 7 Shows pNUT, an example E.coli expression vector used to evaluate fusion reagent clones in vitro.

### DETAILED DESCRIPTION

Unless defined otherwise, all technical and scientific terms used herein have the same meaning as is commonly understood by one of skill in the art to which this invention belongs. All publications and patents referred to herein are incorporated by reference.

Definitions

Transcription associated biomolecules as used herein refer to endogenous compounds that are directly or indirectly associated with transcriptional regulation including but not limited to transcription factors, effectors, ligands, hormones, nuclear hormone receptors, DNA binding domains of nuclear hormone receptors, tumor associated proteins, protein kinases, protein phosphatases, GTP binding proteins, adaptor proteins, secondary messengers of intracellular signaling molecules, and proteins derived from etiological agents.

Regulation of transcription as used herein refers to down regulation via repression, neutralization, or sequestration of transcription associated biomolecules; as well as up regulation via neutralization or sequestration of a repressor—or transcriptional activation via a trans-activation region.

An sFv library as used herein refers to a comprehensive population of V_{L} and V_{H} immunoglobulin variable domains connected by a short flexible peptide linker.

DNA construct library as used herein refers to an sFv library cloned into an expression vector construct such that representative single chain monoclonal antibodies may be expressed in heterologous host cells.

Expression vector as used herein refers to nucleic acid vector constructions which have components to direct the expression of heterologous peptide coding regions including gene fusions of the present invention through accurate transcription and translation in eukaryotic cells. Effective eukaryotic expression vectors usually contain a promoter to direct eukaryotic polymerases to transcribe the heterologous coding region, a cloning site at which to introduce the heterologous coding region, and usually polyadenylation signals. Effective eukaryotic expression vectors include but are not limited to plasmids, retroviral vectors, viral and synthetic vectors.

Gene fusions as used herein refer to nucleic acid sequences derived from different sources, including synthetic sequences which encode amino acid sequences of proteins, that are joined in the same translational reading frame to create one transcriptional unit resulting in a single mRNA transcript which is translated into a chimeric protein. Gene fusions may include a nucleic acid region between, and/or 5′ to, and/or 3′ to the nucleic acid sequences derived from different sources as a linker region or other residue encoding region. Gene fusions of the present invention include those which encode chimeric proteins which differ in polarity in regard to the C- and N-terminal domains. Gene fusions include the joined heterologous nucleic acid coding regions integrated into an effective eukaryotic expression vector for accurate transcription and translation upon introduction into heterologous cells, including in vivo.

Single chain monoclonal antibody as used herein refers to V_{L} and V_{H} immunoglobulin variable domains connected by a short flexible peptide linker which is capable of complexing with an antigen. Single chain monoclonal antibody and single chain monoclonal antibody fusion reagent as used herein are intended to also refer to sFv antibody entities in general identified or isolated by the methods described herein.

DNA regulatory sequence as used herein refers to a nucleic acid sequence to which a DNA binding domain (DBD) of a transcription factor binds and is capable of activating transcription when a trans-activator (transcriptional activator) is associated with the DBD.

Antigenic portion of a transcription associated biomolecule as used herein refers to a portion which is sufficient to raise or generate V_{H} and V_{L} regions to create single chain monoclonal antibody fusion reagents of the present invention capable of binding a transcriptional associated biomolecule in vivo.

ITSP as used herein refers to an intracellular targeting signal peptide or intracellular targeting signal including but not limited to a nuclear localization signal, cytoplasmic localization, endoplasmic reticulum localization signal, mitochondria localization signal, and secretory signal.

Direct administration as used herein refers to the direct administration of nucleic acid constructs which encode single chain monoclonal antibody fusion reagents of the present invention or fragments thereof; and the direct administration of the single chain monoclonal antibody fusion reagents of the present invention or fragments thereof, per se; and the in vivo introduction of gene fusions of the present invention preferably via an effective eukaryotic expression vector in a suitable pharmaceutical carrier. Gene fusions of the present invention may also be delivered in the form of nucleic acid transcripts.

Gene therapy as used herein refers to 1) the direct administration of gene fusions of the present invention and 2) the introduction of somatic cells, including cells transformed with gene fusions of the present invention, into the body of a subject.

Transformed eukaryotic cells or heterologous host cells as used herein refer to cells which have gene fusions of the present invention stably integrated into their genome, or episomally present as replicating or nonreplicating entities in the form of linear nucleic acid or transcript or circular plasmid or vector.

A yeast two-hybrid system has been described wherein protein:protein interactions could be detected using a yeast-based genetic assay via reconstitution of transcriptional activators. Fields, S., Song, O., Nature 340:245 (1989). The two-hybrid system used the ability of a pair of interacting proteins to bring a transcription activation domain into close proximity with a DNA-binding site that regulates the expression of an adjacent reporter gene. See also, Mendelsohn, A. R., Brent, R., Curr. Op. Biotech., 5:482 (1994); Phizicky. E. M., Fields, S., Microbiological Rev., 59(1):94 (1995); Yang, M., et al., Nucleic Acids Res., 23(7):1152 (1995); Fields, S., Sternglanz, R., TIG, 10(8):286 (1994); and U.S. Pat. Nos. 5,283,173, System to Detect Protein-Protein Interactions, and 5,468,614, which are incorporated herein by reference.

The structure of immunoglobulin molecules consist of heavy and light chains which are further defined into variable (V_{H} and V_{L}) and constant domains, the combination of which produces an antigen binding region. The variable regions can be further subdivided into framework regions which are fairly conserved among antibodies and hypervariable regions (CDR) which are quite diverse and are important in defining antigen specificity. The smallest single chain antibody fragment which forms an antigen binding site is referred to as an sFv fragment. Based on random combination events of heavy and light chains in any one antibody-producing cell, the potential repertoire of antibody heavy and light chain combinations may be as much as 10¹² or greater. Thus, to sample a large fraction of this repertoire and obtain clones which express an antibody having a desired antigen binding specificity from a recombinant DNA library can be a daunting task.

Methods are needed which facilitate the screening process, thereby enabling DNA sequences which encode antibody molecules of interest, to be more readily identified, recloned and expressed. Were such procedures available, it may become possible to probe an animal's entire antibody repertoire, for example, to obtain an antibody to a preselected target molecule. In this manner the difficulties and labor intensive process of generating monoclonal antibodies, regardless of the species of origin, by conventional hybridization or transformation of lymphoblastoid cells, may be avoided. The present invention fulfills these and other related needs.

Transcriptional activation

Transcription can be activated through the use of two functional domains of a transcription factor, a domain that recognizes and binds to a specific site on the DNA, and a domain that is necessary for trans-activatior. Keegan, et al., Science, 231, 699-704 (1986) and Ma and Ptashne, Cell, 48, 847-853 (1987). The DNA-binding domain functions to position the transcriptional activation domain on the target gene which is to be transcribed. In some cases, these two functions, DNA-binding domain (DBD) and trans-activator (TA) reside on separate proteins. One protein binds to the DNA, and the other protein, which activates transcription, binds to the DNA-bound protein, as reported by McKnight et al,, Proc. Nat'l Acad. Sci. USA, 89, 7061-7065 (1987); another example is reviewed by Curran, et al., Cell, 55, 395-397 (1988).

Transcriptional activation has been studied, for example, using the GAL4 protein of the yeast Saccharomyces cerevisiae. The GAL4 protein is a transcriptional activator required for the expression of genes encoding enzymes for galactose utilization. Johnston, Microbiol. Rev., 51, 458-476 (1987). It consists of an N-terminal DBD domain which binds to specific DNA sequences designated UAS, (upstream activation sequence) and a C-terminal trans-activator (TA) domain containing acidic regions necessary to activate transcription. The N-terninal DBD domain binds to DNA in a sequence-specific manner but fails to activate transcription. The C-terminal TA domain cannot activate transcription because it fails to localize to the UAS. Brent and Ptashne, Cell, 43, 729-736 (1985). However, when both the GAL4 DBD N-terminal domain and C-terminal TA domain are fused together in the same protein, transcriptional activity is induced. Other proteins also function as transcriptional activators by the same mechanism. For example, the GCN4 protein of Saccharomyces cerevisiae as reported by Hope and Struhl, Cell, 46, 885-894 (1986), the ADR1 protein of Saccharomyces cerevisiae as reported by Thukral, et al., Molecular and Cellular Biology, 9, 2360-2369, (1989) and the human estrogen receptor, as discussed by Kumar, et al., Cell, 51, 941-951 (1987) all contain separable domains for DNA binding and for maximal transcriptional activation.

Signal transduction

Cells respond to environmental changes by sensing substances known as ligands and hormones. Signal transduction involves binding of a hormone or ligand to a specific cell surface receptor which initiates a signaling cascade within the cell resulting in the activation of multiple specific protein kinases and/or phosphatases involved in cell growth which in turn influence the activity of specific transcriptional regulatory proteins and associated biomolecules. These signaling pathways converge ultimately at the level of the nucleus to influence specific patterns of gene expression that regulate growth.

Hormonal activation of signal transduction pathways links extracellular signals to intracellular signals commonly referred to as second messengers which eventually influence transcriptional responses resulting in the activation of many cellular genes. Malarkey, K, Belham, CM, Paul, A, Grahm, A, McLees, A, Scott, PH, Plevin, R., Biochem J., 309:361-375, 1995. In this way hormones are able to regulate processes as diverse as homeostasis, reproduction, development, differentiation, mitogenesis and oncogenesis. Transcriptional control of eukaryotic gene expression is tightly regulated by the binding of nuclear factors to control elements. The availability of these factors is determined inter alia by cell type, differentiation state and position in the cell cycle. The identification and characterization of numerous cellular signaling proteins and transcriptional associated biomolecules has progressed rapidly because of technology enabling the introduction of expression plasmids into mammalian cells. Characterization of the effect of transcription associated biomolecules on cellular growth and differentiation and on otherwise tightly regulated gene expression will permit the elucidation and control of many complex signaling pathways.

CREB/ATF Proteins

One major signal transduction pathway in cells is the G-protein receptor coupled activation of adenylate cyclase leading to the generation of the second messenger, cyclic AMP (cAMP), from ATP. This increase in intracellular levels of cAMP is responsible for the activation of Protein Kinase A through a well characterized mechanism whereby the cAMP-bound regulatory subunit (which is inhibitory when bound to the catalytic subunit) dissociates from the active catalytic subunit. The free catalytic subunit is then able to translocate into the nucleus where it phosphorylates transcription factors and other proteins. Nigg, EA, Hilz, H, Eppenberger, HM, Dutly, F. EMBO J 4:2801-2806, 1985. In recent years, many DNA regulatory elements that mediate the transcriptional responses to increases in intracellular cAMP have been identified and characterized. Deutsch, PJ, Hoeffler, JP, Jameson, JL, Lin, JC, Habener, JF. J. Biol. Chem. 263:18466, 1988. The consensus cAMP Responsive Element (CRE) is an octameric palindrome 5′-TGACGTCA-3′. Montminy, MR, Sevarino, KA, Wagner, JA, Mandel, G, Goodman, RH., PNAS. 83:6682,1986. This sequence is very similar to the heptameric phorbol ester (TPA) Responsive Element (TRE) 5′-TGAGTCA-3′. The CRE-Binding protein CREB, was originally cloned from a human placental cDNA library and was found to have structural homology to the jun and fos proteins that are known to bind and mediate transcriptional responses through the TRE sequence. Hoeffler, JP, Meyer, T, Yun, Y, Jameson, J, Habener, JF., Science. 257:680-682, 1988. Since the original cloning of CREB, multiple related members of a family of CREB/ATF (Activating Transcription Factor) proteins have been cloned and characterized. Meyer, TE, Habener, JF., Endocrine Reviews, 14:269-290, 1993. These proteins share the ability to bind to consensus CRE sequences, as well as sharing the common bZIP domain involved.in dimerization and DNA-binding. Of the CREB/ATF proteins characterized to date, only CREB 327/341, ATF-1, and CREM (as well as some isoforms of these factors) have been demonstrated to gain transcriptional activity via a phosphorylation event mediated by protein kinase A.

ATF-2 has been shown to mediate transcriptional activation by the adenoviral E1a protein, however, an endogenous cellular regulator of ATF-2 function has not been identified. Liu, F., Green, MR., Cell, 61:1217-1224, 1990. ATF-2 most likely interacts with endogenous cellular proteins, in a fashion similar to its interaction with the adenoviral E1a protein, to form transcriptional complexes that regulate cell cycle-dependent gene expression.

The structure/function relationships of CREB/ATF proteins and transcription factors in general have been of major research interest within the last decade because of their key importance in cellular regulation. Many of the studies to characterize these proteins have been done in in vitro model systems which measure protein:DNA or protein:protein interactions. Interpretation of the results of these types of studies has been tempered by the obvious limitations of the in vitro systems. Attempts to study functional corellates in vivo have relied predominantly on transient or stable expression of transcriptional effectors. These approaches have suffered from the obvious shortcomings of overexpressing the transcriptional effectors and risking non-physiologically relevant binding and activation from promoters due to straight competition at less optimal binding sequences.

To exemplify the method of the invention, several endogenous proteins that are components of these CREB/ATF complexes are employed.

Pertaining to the invention

The present invention provides a method for screening DNA construct libraries which encode single-chain fragments of immunoglobulin variable domains (sFv's), for those with high affinity for desired antigens in vivo using the activity of a transcriptional activator. More specifically, the present invention is directed to a method for isolating single-chain fragments of immunoglobulin variable domains capable of targeting transcription associated biomolecules in vivo. The present invention is also directed to monoclonal antibody fusion reagents that regulate transcription in vivo.

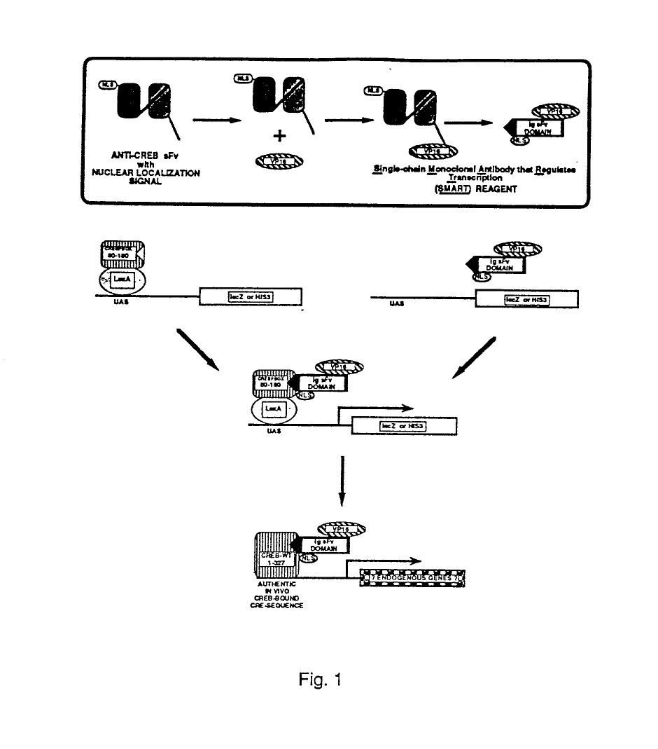

The invention combines the utility of a genetic screening protocol with the specificity of novel vectors that express antigen binding domains of immunoglobulins to target endogenous transcriptional associated regulatory proteins in vivo. The resulting single chain monoclonal antibody fusion reagents may target endogenous DNA-bound transcriptional regulatory proteins in the context of the chromatin present in the promoter region of the target gene of interest. These antibody fusion reagents enable the ability to measure the level of mRNA from the gene under control of the targeted protein (antigen) as well as the determination of which member of diverse families are present and bound to regulatory sequences. See FIG. 1.

Method

I. A peptide DNA binding domain (DBD) of a transcription factor or activator is used in the present invention which binds a corresponding DNA regulatory sequence in vivo and has a corresponding trans-activation peptide to activate transcription of a gene under the control of the DNA regulatory sequence.

In one embodiment of the invention, a nucleic acid fragment which encodes a peptide DBD of a transcription factor is cloned into an expression vector to yield a construct 1 such that the DBD may be expressed in a bio-active form and bind the corresponding DNA regulatory sequence binding site in a heterologous host cell.

A nucleic acid fragment which encodes an “antigenic” portion of a peptide bait (X), preferably a transcription associated biomolecule, is cloned into construct 1, fused in sense orientation in the same translation reading frame, preferably 3′ to, and adjacent to the nucleic acid fragment which encodes the DBD of a transcription factor, to yield a construct 2 such that a resulting chimeric DBD/bait antigen (X) may be expressed in a bio-active form and bind the corresponding DNA regulatory sequence in a heterologous host cell.

The resulting chimeric DBD/bait antigen (X) hybrid peptide encoded by the vector construct 2 is capable of binding a DBD-corresponding transcriptional regulatory nucleic acid sequence in vivo. In one example the DBD-corresponding transcriptional regulatory sequence controls a reporter gene in vivo. Construct 2 accordingly, encodes the “bait”, peptide antigen (X), selection component for the method of the invention.

II. An sFv library (Y) (V_{L} and V_{H} immunoglobulin domains connected by a short flexible peptide linker) as a component for screening is cloned into a separate DNA construct expression vector to yield a construct 3 such that a single chain monoclonal antibody (fusion reagent) may be expressed in bio-active form and bind the corresponding antigen in a heterologous host cell.

A nucleic acid fragment which encodes a trans-activation peptide, is cloned into construct 3, preferably fused in sense orientation in the same translation reading frame, preferably 3′ and adjacent to the nucleic acid fragment which encodes a single chain monoclonal antibody, to yield a construct 4 such that a resulting chimeric sFv (Y)/trans-activation peptide may be expressed in bio-active form and bind the corresponding antigen in a heterologous host cell. The corresponding antigen is most preferably a transcription associated biomolecule.

The hybrid peptide encoded by the vector construct 4 is comprised of an immunoglobulin variable region (Y) as the component for screening, covalently attached to a transactivation peptide (TA) which is capable of activating a reporter gene in vivo via the DNA binding domain of the hybrid peptide of construct 2.

A. The method may comprise fusing at least one nucleic acid fragment which encodes an intracellular targeting signal in the same translation reading frame to the nucleic acid fragment which encodes the single chain monoclonal antibody in construct 4, to yield a modified construct (5) such that a resulting single chain monoclonal antibody fusion reagent may be expressed in bio-active form and bind the corresponding antigen in a heterologous host cell.

Another preferred embodiment is accomplished by fusing a nucleic acid fragment which encodes an intracellular targeting signal in the form of a nuclear localization signal (NLS) in the same translation reading frame to the nucleic acid fragment which encodes the single chain monoclonal antibody in construct 4, to yield an alternate modified construct (5) such that a resulting single chain monoclonal antibody fusion reagent may be expressed in bio-active form and bind the corresponding antigen in a heterologous host cell nucleus.

III. The method includes providing a heterologous host cell, preferably a yeast cell, most preferably Saccharomyces cerevisiae or Schizosaccharomyces pombe. The host cell contains a detectable gene under transcriptional control of a DNA regulatory sequence binding site corresponding to the DBD encoded by construct 2, such that the detectable gene expresses a detectable protein when the detectable gene is transcriptionally activated when the trans-activation peptide encoded by construct 4 is brought into sufficient proximity to the DBD encoded by construct 2.

A peptide DNA binding domain (DBD) of a transcription factor is used in the present invention which binds a corresponding DNA regulatory sequence in vivo and has a corresponding trans-activation peptide to activate transcription of a gene under the control of the DNA regulatory sequence.

Host cells comprising an assayable reporter gene as the detectable gene under transcriptional control of the DNA regulatory sequence which corresponds to the DBD of construct 2 are transformed with constructs 2 and 4 to screen for chimeric antibody (Y)/transcriptional activator fusion reagents of construct 4 with strong affinity for DBDlantigen (X) fusion reagents of construct 2 that are capable of activating transcription of the reporter detectable gene in vivo.

Therefore if the immunoglobulin variable region (Y) of a construct 4 hybrid peptide has strong affinity for the peptide antigen (X) of the construct 2 hybrid peptide, the transactivation peptide (TA) will be brought in effective proximity to bioactivate the DBD and hence “turn on” the reporter detectable gene in vivo.

Alternate method

A preferred embodiment of the present invention for screening a DNA construct library for a single chain monoclonal antibody fusion reagent capable of binding a transcriptional associated biomolecule in vivo comprises providing an expression construct (1) which encodes a peptide DBD of a transcription factor and comprises a cloning site for fusing a nucleic acid fragment which encodes an antigenic portion of a transcriptional associated biomolecule in the same translation reading fiame of the nucleic acid fragment which encodes the DBD of a transcription factor, to yield a construct (2). The method further comprises providing a DNA construct (3) which encodes a trans-activation peptide and comprises a cloning site for fusing an sFv library in the same translation reading frame of the trans-activation peptide, to yield a construct (4) such that a resulting chimeric sFv/trans-activation peptide may be expressed in bio-active form and bind a transcriptional associated biomolecule in a heterologous host cell.

The method further comprises providing a heterologous host cell, harboring a detectable gene under transcriptional control of the DNA regulatory sequence binding site corresponding to the DBD encoded by construct 2, for introducing constructs 2 and 4 into the heterologous host cell, such that both constructs may be expressed.

The method further comprises identifying a DNA construct 4 which encodes a single chain monoclonal antibody reagent capable of binding the transcriptional associated biomolecule in vivo by selecting for expression of the detectable gene.

A preferred method for screening a DNA construct library for a single chain monoclonal fusion reagent capable of binding a transcriptional associated biomolecule in vivo comprises providing pVP16Zeo (ATCC deposit #______) as DNA construct 3. Another contemplated embodiment of the method provides a human sFv library integrated into DNA construct 3, preferrably pVP16Zeo. Another contemplated embodiment of the method provides primers for human sFv library construction. In one embodiment primers may be selected from the group consisting essentially of SEQ ID NOs: 3-86 as described infra. See Tables I-III.

Kit

Another embodiment of the present invention is a kit for screening a DNA construct library for a single chain monoclonal antibody fusion reagent capable of binding a transcriptional associated biomolecule in vivo; comprising in a container: an expression construct (1) which encodes a peptide DBD of a transcription factor such that the DBD may be expressed in a bio-active form and bind a corresponding DNA regulatory sequence binding site in a heterologous host cell. The expression construct 1 further comprises a cloning site for fusing a nucleic acid fragment which encodes an antigenic portion of a transcriptional associated biomolecule into the construct 1, in the same translation reading frame of the nucleic acid fragment which encodes the DBD of a transcription factor, to yield a construct (2). The kit further comprises a DNA construct (3) which encodes a trans-activation peptide and comprises a cloning site for fusing an sFv library in the same translation reading fiame of the trans-activation peptide, to yield a construct (4) such that a resulting chimeric sFv/trans-activation peptide may be expressed in bio-active form and bind a transcriptional associated biomolecule in a heterologous host cell.

The kit further comprises a heterologous host cell harboring a detectable gene under transcriptional control of the DNA regulatory sequence binding site corresponding to the DBD encoded by construct 2, for introducing constructs 2 and 4 into the heterologous host cell harboring a detectable gene under transcriptional control of the DNA regulatory sequence binding site corresponding to the DBD encoded by construct 2, such that both constructs may be expressed.

The kit further comprises a means for identifying a DNA construct 4 which encodes a single chain monoclonal antibody reagent capable of binding the transcriptional associated biomolecule in zivo by selecting for expression of the detectable gene. A means for identifying such a DNA construct include items such as prepackaged selective media and/or protocols described herein as to how to identify positive constructs.

A preferred kit for screening a DNA construct library for a single chain monoclonal fusion reagent capable of binding a transcriptional associated biomolecule in vivo comprises pVP16Zeo (ATCC deposit #______) as DNA construct 3. Another contemplated embodiment of the kit provides a human sFv library integrated into DNA construct 3, preferrably pVP16Zeo. Another contemplated embodiment of the kit provides primers for human sFv library construction. In one embodiment primers may be selected from the group consisting essentially of SEQ ID NOs: 3-86 as described infra. See Tables I-III.

Alternate embodiments

Single chain monoclonal antibody fusion reagent as used herein also refers to truncated forms wherein the TA region is deleted, especially for therapeutic use for regulating the function of a transcriptional associated biomolecule in vivo by means of neutralization/sequestration of the biomolecule. Preferred embodiments therefore comprise fusing at least one nucleic acid fragment which encodes an intracellular targeting signal in the same translation reading frame to the nucleic acid fragment which encodes the single chain monoclonal antibody in construct 4, and deleting the TA, to yield a modified construct (6) such that a resulting single chain monoclonal antibody fusion reagent may be expressed in bio-active form and bind the corresponding antigen in a heterologous host cell. These types of reagents may also be used to “track” intracellular transport of various characteristic sFv targets.

Single chain fusion reagents of the present invention may comprise a transcriptional repressor (TR) or a repressor interacting domain (RID) instead of a transcriptional activator (TA). Embodiments of this type are capable of repressing the expression of a reporter gene in vivo. Therefore a further object of the invention is to provide a therapeutic method for regulating the transcription of a gene in vivo by means of transcriptional repression. See. e.g., Ayer, D. E., et al., Cell, 72: 211 (1993); Henriksson, M., et al., Adv. Cancer Res., 60:109 (1996); Hurlin, et al., EMBO J., 14:5646; Gilbert, W., et al., PNAS, 56:1891 (1966); Ptashne, M., PNAS, 57:306 (1967); Pabo, C. O., et al., Nature, 298:443 (1982); Steitz, T. A., et al., PNAS, 79:3097 (1982).

The invention is not limited to the particular steps or constructs described herein. Preferred elements of the invention comprise a construct which encodes a DBD fused to an antigenic portion of a transcription associated biomolecule which can be expressed in a host cell; a construct population which encodes an sFv library (preferably V_{L} and V_{H} immunoglobulin domains connected by a short flexible peptide linker) fused to a transcriptional activator which can be expressed in a host cell; and a host cell which harbors a detectable gene under transcriptional control of the DNA regulatory sequence binding site corresponding to the DBD.

The heterologous host cell includes but is not limited to a strain or a cell line having a selectable marker gene or reporter gene as the detectable gene. A heterologous host cell having a selectable marker gene or reporter gene as the detectable gene may be transformed sequentially with constructs 2 and 4. Alternatively, for instance, separate haploid yeast strains, one or both having a selectable marker gene or reporter gene as the detectable gene, each harboring construct 2 or 4, may be mated and diploids harboring both constructs selected by methods which are well known to those skilled in the art. Herskowitz, I., Microbiol. Rev., 52:536 (1988); Sherman, F., et al., Methods in Yeast Genetics, CSH, NY (1979).

Basically, the antigen (X) fusion construct is used as a “bait” to screen for single chain monoclonal antibody reagents that, in a preferred embodiment, regulate transcription in vivo. Any peptide antigen (X) may be used as the “bait” for screening antibody fusion reagents described herein for specificity. In a preferred embodiment of the present invention (X) is a transcriptional associated protein. The system operates by screening for antibody fusion reagents which have strong affinity for transcription associated peptides, which reagents are identified by their ability to enhance transcription of a reporter gene. Accordingly, this method may be used to identify and produce single chain monoclonal antibody reagents that regulate transcription in vivo.

Preferred embodiments of the single chain fusion reagents have the general structures:

*NH₂—V_{H}—linker—V_{L} —transcriptional activator—COOH* (I)

*NH₂—ITSP—V_{H}—linker—V_{L}—transcriptional activator—COOH* (II)

*NH₂—ITSP—V_{H}—linker—V_{L}—ITSP—transcriptional activator—COOH* (III)

*NH₂—V_{H}—linker—V_{L}—COOH* (IV)

*NH₂—ITSP—V_{H}—linker—V_{L}—COOH* (V)

*NH₂—ITSP—V_{H}—linker—V_{L}—ITSP—COOH* (VI)

The V_{H} and V_{L} regions of the single chain fusion reagents of the present invention may be reversed, i.e. V_{H}—linker—V_{L} or V_{L}—linker—V_{H}. ITSP as used herein refers to an intracellular targeting signal peptide or intracellular targeting signal.

Antibody reagents

Single chain monoclonal antibody reagents identified by the method of the invention are single chain peptides comprised of heavy V_{H} and light V_{L} immunoglobulin variable domains connected by a flexible linker and a transcriptional activator peptide (TA) fused to the C-terminus; which are capable of binding transcription associated biomolecules in vivo. Preferred embodiments of the single chain monoclonal antibodies identified by the method of the invention are fusion reagents further comprised of a peptide intracellular targeting signal, most preferably a nuclear localization sequence (NLS), fused to the N-terminus or C-terminus, or both, of the immunoglobulin variable domains. Accordingly, preferred peptide fusion reagents are comprised of nuclear localization signal(s) fused to immunoglobulin regions with strong affinity to a transcriptional associated biomolecule, and a C-terminal transcriptional activator peptide (TA) for the regulation of transcription in vivo.

Modified single chain fusion reagents of the present invention may be comprised of only the heavy V_{H} and light V_{L} immunoglobulin variable domains connected by a flexible linker—(due to a deleted transcriptional activator peptide (TA))—which are capable of binding transcription associated biomolecules in vivo. Other modified single chain fusion reagents of the present invention may be comprised of merely a peptide intracellular targeting signal fused to the N-terminus or C-terminus, or both, of the immunoglobulin variable domains—(due to a deleted transcriptional activator peptide (TA))—which are capable of binding transcription associated biomolecules in vivo. Still other modified single chain fusion reagents of the present invention may comprise a transcriptional repressor (TR) or a repressor interacting domain (RID) in place of a transcriptional activator (TA).

Preferred embodiments of the single chain monoclonal antibody fusion reagents consist of an antibody light chain variable domain (V_{L}) and heavy chain variable domain (V_{H}) connected by a short flexible linker, preferably a peptide [(Gly)₄Ser]₃ which allows the molecule to assume a conformation that is capable of binding an antigen. Nicholls, PJ, Johnson, VG, Blanford, MD, Andrew, SM., J. Immunol. Methods, 165:81-91, 1993. Most preferably there is a short flexible linker between the two immunoglobulin variable domains, e.g., V_{L}—[(Gly)₄Ser]₃—V_{H}; or V_{H}—[(Gly)₄Ser]₃—V_{L}.

Intracellular targeting

A characteristic amino-terminal transient signal sequence of transported protein is a common principle in major organelle systems that transport proteins across a membrane. Schatz, G., Dobberstein, B., Science, 271 (5255):1519 (1996); Gorlich, D., et al., Science, 271(5255): 1513 (1996). Intracellular targeting of specific antibody reagents of the present invention by directing expression of the antibody reagent to different cellular compartments enables selective targeting and the corollary inhibition, sequestration or neutralization of a molecule's bioactivity. Therefore, preferred embodiments of single chain monoclonal antibody fusion reagents of the present invention have an intracellular targeting signal, most preferably fused to the N-terminus or C-terminus, or both, of the immunoglobulin variable domain, to enable targeting of specific antibodies to specific cellular compartments with the aim of complexing with characteristic antigens. Fusion reagents of the present invention which regulate trascription are preferably comprised of at least one nuclear localization sequence (NLS).

The method described herein can be used to screen for and design fusion reagents which target a wide variety of transcription associated biomolecules including ones that norinally reside in the nucleus, cytoplasm, mitochondria, extracellular, or are peripherally associated with membranes. The method described herein can be used to screen for and prepare fusion reagents which target nuclear expression transcription associated biomolecules for transcription enhancement, repression, or anti-transcription factor function. The method described herein can be also used to design and prepare fusion reagents which target cytoplasmic biomolecules for the production of anti-signaling molecules. The method described herein can be used to design and prepare fusion reagents which target endoplasmic reticulum expression to utilize fusion reagents to prevent secretion of specific proteins. The method described herein can be used to design and prepare fusion reagents which target mitochondrial expression to produce, for instance, anti-cytochrome C oxidase fusion reagents. The method described herein can be used to design and prepare fusion reagents which target secreted expression in an expression system to produce the fusion reagents.

Embodiments for specific intracellular targeting of antibody fusion reagents

Targeting vectors may direct expression of single chain antibodies to intracellular compartments including the cytoplasm, nucleus, endoplasmic reticulum, and the mitochondria, as well as secretory. The example targeting signals (described by Biocca, S, Ruberti, F, Tafani, M, Pierandrei-Amaldi, P, Cattaneo. Biotech. 13:1110-1115, 1995) are each shown to be functional and to thereby target the single chain antibody to the proper compartment. Successful targeting has been demonstrated in the endoplasmic reticulum, the mitochondria, the cytosol and nucleus with versions of the same single chain antibody, all of which are clearly capable of recognizing and in some cases neutralizing their target antigens.

One cytoplasmic expression embodiment allows cloning of the antibody region in frame with a C-terminal myc epitope tag. In this manner anti-“signaling” fusion reagents may be expressed in the cytosol to arrest signal transduction. Anti-Ras fusion reagents, for example are a contemplated embodiment of this aspect of the invention. One alternative for the cytosolic expression of antibody reagents is to incorporate a CAAX tag to anchor the sFv in the lipid membrane. This approach is useful for sequestering intracellular signaling molecules and therefore inhibit their function.

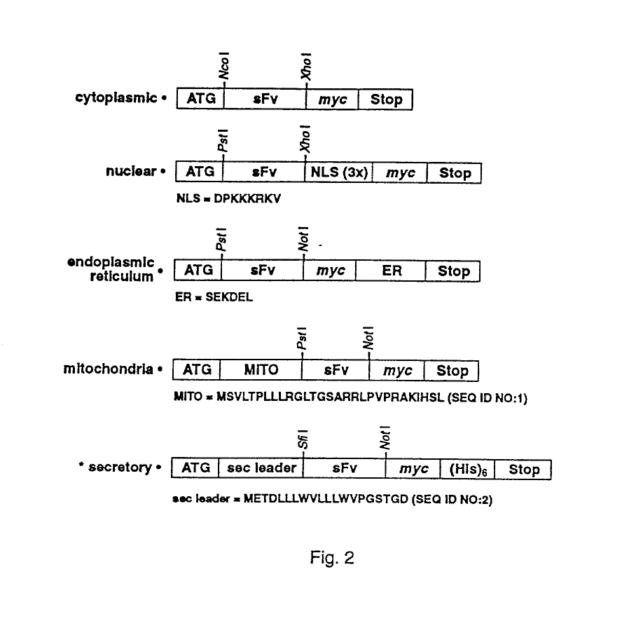

Another embodiment of the present invention is nuclear expression for anti-transcription factor single chain monoclonal antibody fusion reagents. A nuclear-targeting version of an expression vector (FIG. 2) facilitates cloning of the immunoglobulin domain with 3 repeats of the nuclear localization signal (NLS) derived from SV40 T antigen (DPKKKRKV) and a myc epitope tag at the C-terminus. Biocca, S, Nueberger, M S, Cattaneo, A. Embo J. 1:101-108, 1990.

Targeting of single chain antibody fusion reagents of the present invention to the endoplasmic reticulum is a contemplated embodiment to prevent secretion of specific proteins. A presently available endoplasmnic reticulum (ER) targeting vector allows for cloning of the antibody region in frame with a myc epitope tag followed by an ER retention signal (SEKDEL). Munro, S, Pelham, RB. Cell 48:899-907, 1987. The utility of this embodiment is to prevent secretion of a protein that is normally secreted by sequestration/neutralization and/or retaining the target/fusion reagent complex in the endoplasmic reticulum. Anti-erbB2 and anti-VEGF are embodiment fusion reagents to block secretion of a transmembrane protein (epidermal growth factor (EGF) receptor with anti-erbB2) and a secreted protein (vascular endothelial growth factor (VEGF) with anti-VEGF).

A mitochandrial expression vector enables another embodiment which facilitates cloning of the antibody domain in frame with a 5′ N-terminal presequence of the subunit VIII of human cytochrome C oxidase (COX8.21) and a C-terminal myc epitope tag to facilitate mitochondrial targeting of an anti-cytochrome C oxidase embodiment. See FIG. 2. The mitochondrial target signal is 25 amino acids of presequence and the first 4 amino acids of mature human cytochrome oxidase: MSVLTPLLLRGLTGSARRLPVPRAKIHSL (SEQ ID NO:1). Rizzuto, R, Simpson, AWM, Brini, M, Pozzan, T. Nature 358:325-327, 1992.

A secretory expression vector enables another embodiment wherein the target signal is 20 amino acids: METDLLLWVLLLWVPGSTGD (SEQ ID NO:2).

In addition to the specific targeting vectors described herein a vector is also contemplated which will allow expression of the antibody fusions with green fluorescent protein (GFP) as a C-terminal tag. This will allow for visual tracking of their intracellular expression . GFP is a 238 amino acid protein which can be easily visualized by fluorescent microscopy. GFP stably emits green light when excited by blue light and unlike many bioluminescent proteins, requires no exogenous substrates or cofactors for fluorescence making it an ideal marker for monitoring the traffic of proteins in living organisms. Cubitt, AB, Heim, R, Adams, SR, Boyd, AE, Gross, LA, Tsien, RY. TIBS 20:448-455, 1995.

All targeting signals described herein have been shown to be functional.

DNA binding domain

Any DBD may be used for fusion to an antigen as part of the selection component of the invention. DNA binding domains are preferred which have a corresponding transactivation peptide for fusion with the immunoglobulin variable region (Y) screening component of the invention.

The GAL4 DNA binding domain may be used for instance that is derived from the yeast Gal4 protein. Chien, C. T., et al.,PNAS, 88:9578 (1991). Anther embodiment described herein uses E.coli. LexA as a DBD in a hybrid construct. Vojtek, A.B., et al., Cell, 74:205. The DNA binding domain and the transcriptional activation domain may be from any transcriptional activators including but not limited to GAL4, GCN4 and ADR1.

Antigen X (antigenic portion of a transcription associated biomolecule)

Any peptide coding region may be used as an antigen component for selection in the present invention. Preferred embodiments are transcription associated biomolecules which include transcription factors, intercellular signaling molecules, intracellular signaling molecules, second messengers, hormones, ligands, receptors, nuclear hormone receptors, DNA binding domains of nuclear hormone receptors, tumor associated proteins, protein kinases and/or phosphatases, GTP binding proteins, adaptor proteins, secondary messengers of an intracellular signaling molecules, and proteins derived from etiological agents.

Preferred embodiments of the “bait”, peptide antigen (X), selection component of the invention for screening sFvs include multiple members of the CREB/ATF transcription factor family including but not limited to ATF-1, ATF-3, ATF-4, ATF-6, and CREM. Lalli, E., et al., J. Biol. Chem., 269:17359 (1994); Haebner, J., Mol. Endo., 4:1087 (1990).

Other preferred embodiments of the “bait”, peptide antigen (X), selection component of the invention for screening sFvs include but are not limited to the intracellular signaling molecules Ras, Grb2, PLCγ, Syp, P13K, MAPK, JNK as well as the DNA binding domains of nuclear hormone receptors including but not limited to the androgen receptor (AR), thyroid hormone receptor (TR), glucocorticoid receptor (GR). Kazlauskas, A., Current Biology (Curr. Op. in Gen. and Dev.), 4:5 (1994); Cano, E, et al., Trends Biochem. Sci., 20:117 (1995); Quigley, C. A., et al., Endocrine Rev., 16:271 (1995); Chatterjee, V. K., et al., Cancer Surv., 14:147 (1992); Bodine, P. V., et al., Receptor, 1:83 (1990).

Protein derived from an etiological agent may be used. Proteins that are native or derived from viral, bacterial or unicellular or multicellular pathogens or tumor associated proteins. Nucleic acid fragments which encode proteins derived from etiological agents used to construct genetic fusions of the present invention include but are not limited to those which encode, for instance, HIV proteins, proteins from malaria causative organisms, including Plasmodium falciparum, Hepatitis A and B, respiratory syncytial virus RSV (pediatric pathogen), HIV, Junin virus, herpes simplex virus (HSV I and II), rubella, cytomegalo virus (CMV), Varicella-Zoster virus (VZV), Epstein-Barr virus (EBV), Measles, Hantaviruses, Dengue virus, Ebola virus, and tumor-associated antigens.

Transactivation peptide (TA)

The transactivation peptide may be derived from the transcription factor GAL4. Chien, C. T., et al., PNAS, 88:9578 (1991). Other embodiments described herein, for example, may use the Herpes simplex virus VP16 protein or c-Fos as a transactivation peptide. Dalton, S., Treisman, R., Cell, 68:597 (1992); Rauscher, F. J. I., et al., Science, 240:1010 (1988). Other embodiments of transactivation domains may be used including B42—an activation domain derived from E.coli which is also functional in yeast. Ma, J., Ptashne, M., Cell, 51:113 (1987). Any functional acidic sequences or domains that transactivate may be used with the present invention.

A preferred embodiment of the invention is an immunoglobulin variable region cDNA/VP16 TA fusion library so one can screen for unknown antibody fusion reagents that interact with a LexA DBD/protein X fusion of interest.

An embodiment described herein comprises a first hybrid construct encodes a LexA DBD/protein X “bait” fusion, while a second hybrid construct encodes an immunoglobulin variable region library/VP16 TA fusion for screening. Expression constructs which encode these hybrid peptides are transformed into yeast with reporter genes (LacZ and His3) whose regulatory regions contain the UAS LexA binding site. Positive interactions are detected by selection on His− plates as well as a second β-gal screen.

The antibody fusion reagent in another embodiment has an engineered nuclear localization signal (NLS) from the SV40 T-Ag incorporated into the construct to target the VP16 fusion to the nucleus. The two hybrid proteins are transformed into a Saccharomyces cerevisiae strain which has two reporter genes (lacZ and HIS3) whose regulatory regions contain the UAS LexA binding site.

Vectors

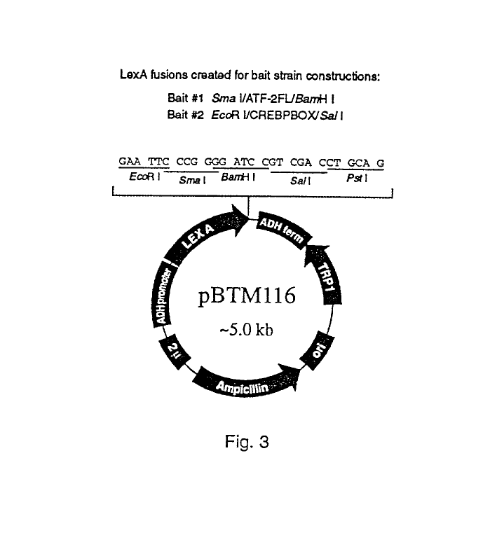

Peptide antigen (X) “bait” strains may be constructed as LexA DBD fusions in pBTM116 for example, to screen an antibody fusion reagent library. The pBTM116 yeast expression plasmid (ATCC access #______) (FIG. 3) contains a Trp1 gene for selection in yeast and the DBD of Lex A with a downstream polylinker to allow generation of Lex A DBD/antigen X (“bait”) fusion proteins. Vojtek, A. B., Hollenberg, S. M., Cooper, J. A., Cell, 74:205 (1993).

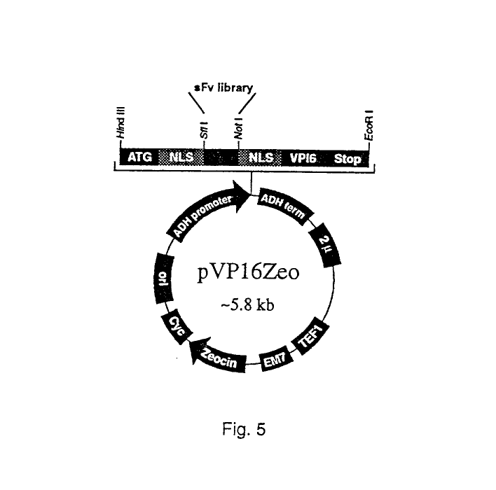

Novel vectors that express the single chain monoclonal antibody fusion reagents are also embodiments of the present invention. The vector pVP16Zeo, described infra, is a most preferred embodiment (FIG. 5) (ATCC access #______). The pVP16Zeo library expression vector is most preferred for the construction and screening of single chain monoclonal antibody fusion reagent libraries, comprising zeocin selection to facilitate the isolation and production of single chain monoclonal antibody fusion reagents in yeast and E.coli. Generally, relatively small cloning vectors (under 5 kb) which have a convenient multiple cloning site as well as functional promoter (e.g. yeast ADH promoter) to drive expression of the heterologous sequence as well as efficient termination signals for 3′ mRNA processing—are preferred for ease of manipulation in library construction. Zeocinr is preferred as a dual selectable marker in yeast and E.coli.

Detectable reporter genes include but are not limited to E.coli LacZ and selectable yeast genes such as HIS3 and LEU2. Fields, S., Song, O., Nature 340:245 (1989); Durfee, T., et al., Genes Dev., 7:555 (1993); Zervos, A. S., et al., Cell, 72:223 (1993). The reporter gene function can be served by any of a large variety of genes, such as genes encoding drug resistance or metabolic enzymes. Genes may be studied in vivo wherein mRNA transcripts are detected via Northern blot analysis as well as other assays including PCR methods to determine transcription and gene expression well known to those skilled in the art.

Cloning V_{H} and V_{L} regions

A variety of techniques exist for preparing the sFv library, which is preferably prepared from cDNA. See, e.g., Sambrook et al., Molecular Cloning, A Laboratory Manual, 2nd ed., Cold Spring Harbor Laboratory Press, Cold Spring Harbor, N.Y., 1989, which is incorporated herein by reference. RNA and cDNA may be prepared from spleen cells from unimmunized animals, from animals immunized with antigens or haptens of interest, hybridoma cells, or lymphoblastoid cells, for example. The use of spleen cells from unimmunized animals provides a better representation of the possible antibody repertoire, while spleen cells from immunized animals are enriched for sequences directed against epitopes of the immunizing antigen or haptens. The cells may be obtained from a variety of animal species, such as human, mouse, rat, lagomorpha, equine, bovine, avian, etc., the selection often dependent on the antibody of interest and the use for which it is intended.

Amplification of sequences representing messenger RNA (mRNA) isolated from cells of interest, such as spleen or hybridoma cells, may be performed according to protocols outlined in, e. g., U.S. Pat. No. 4,683,202, Orlandi, et al. Proc. Natl. Acad. Sci. USA 86:3833-3837 (1989), Sastry et al., Proc. Natl. Acad. Sci. USA 86:5728-5732 (1989), and Huse et al. Science 246:1275-1281 (1989), each incorporated herein by reference. See also, PERKIN ELMER-Biotechnology Catalog and PCR Bibliography, Norwalk Conn. Oligonucleotide primers useful in amplification protocols may be unique or degenerate or incorporate inosine at degenerate positions. Thus, for multi-chain immunoglobulins, primers would be generally used for amplification of sequences encoding the variable regions of both the heavy and light chains. Restriction endonuclease recognition sequences may be incorporated into the primers to allow for the cloning of the amplified fragment into a vector in a predetermined reading frame for expression.

Polymerase chain reaction (PCR)-based systems are used to practice the present invention that allow the isolation of immunoglobulin variable regions using mRNA isolated from cells including human and murine spleen cells or peripheral blood lymphocytes in addition to murine hybridoma cells. Coloma, MJ, Hastings, A, Wims, LA, Morrison, SL. J. Immunol. Methods 152:89-104, 1992; Marks, JD, Hoogenboom, HR, Bonnert, TP, McCafferty, J, Griffiths, AD, Winter, G. Mol. Biol. 222:581-597, 1991. The variable domains can be derived from other sources. Current methods allow PCR primers to be designed such that immunoglobulin variable regions can be directly amplified without prior knowledge of their sequence. Coloma, MJ, Larrick, JW, Ayala, M, Gavilondo-Cowley, JV. BioTechniques 11:152, 1991. Once isolated, these variable regions can be manipulated in many different ways to produce biologically active molecules.

Methods are generally known for directly obtaining the DNA sequence of the variable regions of any immunoglobulin chain by using a mixture of oligomer primers and PCR. For instance, mixed oligonucleotide primers corresponding to the 5′ leader (signal peptide) sequences and/or FR1 sequences and a conserved 3′ constant region primer have been used for PCR amplification of the heavy and light chain variable regions from a number of human antibodies directed to, for example, epitopes on HIV-I (gp 120, gp 42), digoxin, tetanus, immunoglobulins (rheumatoid factor), and MHC class I and II proteins (Larrick et al. (1991) Methods: Companion to Methods in Enzymology 2:106-110). A similar strategy has also been used to amplify mouse heavy and light chain variable regions from murine antibodies, such as antibodies raised against human T cell antigens (CD3, CD6), carcino embryonic antigen, and fibrin (Larrick et al. (1991) BioTechniques 11: 152-156).

To generate single chain antibodies (sFv's), MRNA is isolated from the cell line or tissue of interest. The mRNA is then used as a template, usually with a synthetic oligo dT primer, for the synthesis of single stranded cDNA. The resulting single stranded cDNA is then used to generate the light chain product.

Messenger RNA may be isolated from any cell or tissue type including mature B cells of, peripheral blood cells, bone marrow, established hybridomas, or spleen preparations, using standard protocols. First-strand CDNA is synthesized using primers specific for the constant region of the heavy chain(s) and each of the kappa and lambda light chains.

Since the heavy chain message is considerably larger and it is essential that the 5′ end of the message encoding the V_{H} is incorporated in the cDNA, the mRNA used to generate the heavy chain variable region cDNA is primed with a constant region specific primer.

The linkered variable region PCR products are generated using the appropriate primers that have been fused to a sequence that when overlapped with the homologous sequences from the other chain variable region product will encode the [(Gly)₄Ser]₃ linker sequence between the two variable domains.

The linkered variable domain PCR products are gel purified annealed with their con-esponding partner and extended in a recombinant PCR reaction to produce the intact sFv's.

Host cell

While the methods described herein are generally described in yeast cells, e.g. Saccharomyces cerevisiae and Schizosaccharomyces pombe—they are also expected to function similarly in mammalian cells and should be applicable to eucaryotic host cells in general.

Utility

The synthetic antibodies can be used in any and all applications in which antibodies derived from other sources or other means are used.

Affinity purification

The single chain monoclonal antibodies and fusion reagents identified and produced by the methods described herein may be used for the affinity purification of antigenic biomolecules including transcription associated biomolecules, regulators, effectors, intercellular and intracellular signaling molecules, hormones, receptors and ligands by methods well known to those skilled in the art. A single chain monoclonal antibody may be fixed to a solid matrix, e.g. CNBr activated Sepharose according to the protocol of the supplier (Pharmacia, Piscataway, N.J.), and a homogenized/buffered cellular solution containing the molecule of interest is passed through the column. After washing, the column retains only the molecule of interest which is subsequently eluted, e.g., using 0.5M acetic acid or a NaCl gradient.

In vivo transcriptional regulation

Preferred embodiments of the single chain monoclonal antibody fusion reagents of the present invention regulate gene transcription in vivo. Transcription may be regulated via a transcriptional activator (TA) or a transcriptional repressor (TR) or a repressor interacting domain (RID) instead of a transcriptional activator (TA). Single chain monoclonal antibody fusion reagents of the present invention may be devoid of of a transcriptional activator or any other component beyond the immunoglobulin sFv region.

The single chain monoclonal antibody fusion reagents can be used to control the activities of biomolecules including those which regulate gene transcription in vivo. Reagents of the present invention may be used for the neutralization or sequestration of biomolecules including transcriptional associated regulatory biomolecules thereby preventing- or- down-regulating the expression of a gene. Therefore a preferred embodiment is a single chain monoclonal antibody reagent that neutralizes or sequesters a transcriptional associated biomolecule and thus down-regulates transcription in vivo. Another contemplated embodiment of the present invention is a single chain monoclonal antibody reagent that neutralizes or sequesters a transcriptional associated biomolecule. Another contemplated embodiment of the present invention is a single chain monoclonal antibody reagent that neutralizes or sequesters a transcriptional-repressor, and thus up-regulates or enhances transcription in vivo by effectively removing the biological activity of the repressor.

Accordingly, a therapeutic method for regulating the transcription of a gene in vivo by means of transcriptional activation is provided, comprising administering an effective amount of a single chain monoclonal antibody fusion reagent or a portion thereof that targets a transcriptional associated biomolecule in vivo. A therapeutic method is also provided for regulating the function of a transcriptional associated biomolecule in vivo, comprising administering an effective amount of a single chain monoclonal antibody fusion reagent or a portion thereof that targets the specific biomolecule in vivo.

The method of the invention provides for the production and identification of single chain monoclonal antibodies with specificity for transcription associated biomolecules including intracellular signaling molecules which control transcription from a diverse range of signal transduction pathways; including nuclear hormone receptors and DNA binding domains of nuclear hormone receptors. Any peptide coding region may be used as an antigen component for selection in the present invention. Preferred embodiments include transcription associated biomolecules which include transcription factors, intercellular signaling molecules, intracellular signaling molecules, second messengers, hormones, ligands, receptors, DNA binding domains of nuclear hormone receptors, tumor associated proteins, protein kinases and/or phosphatases, and proteins derived from etiological agents.

Transcriptional associated biomolecules contemplated for use with the present invention, include Ras, Grb2, phospholipase Cγ-PLCγ, phosphatidylinositol 3-kinase-P13K, Syp, mitogen activated protein kinase-MAPK, jun kinase-JNK, androgen receptor (AR), thyroid hormone receptor (TR), glucocorticoid receptor (GR), ATF-1, ATF-2, ATF-3, ATF-4, ATF-6, CREB and CREMτ.

A preferred embodiment of the present invention is a single chain monoclonal antibody fusion reagent that enhances transcription or otherwise up-regulates gene transcription in vivo by means of a transcriptional transactivator (TA), for example, fused to the C-terminus of the fusion reagent. In this embodiment the fusion reagent is most preferably targeted to the nucleus via an intracellular targeting signal—a nuclear localization signal (NLS)—and has affinity for a nuclear transcription associated biomolecule thereby favoring proximity for transcriptional activation.

Therapeutic use

Human monoclonal antibodies have considerable potential in the prophylaxis and treatment of viral disease. The present invention is expected to be of value in generating antibodies to be used both in the prophylaxis and treatment of viral infections and in the characterization of the mechanisms of antibody protective actions at the molecular level. The single chain monoclonal antibodies and fusion reagents produced and identified by the methods described herein are contemplated for use as bio-therapeutic immunotherapy and gene regulation in vivo. The single chain monoclonal antibodies may be used to sequester and/or neutralize pathological agents as well as to control transcription of pathological genes through activation, repression, or indirectly-through interaction with transcription associated biomolecules. Single chain monoclonal antibodies and fusion reagents for use against Hepatitis A and B, respiratory syncytial virus RSV (pediatric pathogen), HIV, Junin virus, herpes simplex virus (HSV I and II), rubella, cytomegalo virus (CMV), Varicella-Zoster virus (VZV), Epstein-Barr virus (EBV), Measles, Hantaviruses, Dengue virus,and Ebola virus inter alia are contemplated.

Once specific immunoglobulin variable regions are identified, their coding regions may be used independently (deleted TA) to encode useful reagents or may be fused (as alternatives to their fusion to trans-activators for transcriptional enhancement) to nucleic acids which encode repressors, toxins, enzymes, cytokines, as well as other useful peptide compounds to create novel biopharmaceutics. Neri, D., et al., Engineering Recombinant Antibodies for Immunotherapy, Cell Biophysics, 27:47 (1995); Grifiths, A. D., et al., EMBO J., 13:3245 (1994). U.S. Pat. No. 5,455,030, issued Oct. 3, 1995, Immunotheraphy Using Single Chain Polypeptide Binding Molecules, is herein incorporated by reference. Synthetic antibodies identified from screening can be used for the development of immunotherapeutics. For instance, antibodies can be administered for passive immunization or immunoconjugates which may be used to target tumors or other targets. Single chain monoclonal antibodies with affinity for transcription associated biomolecules are contemplated which are capable of neutralizing or sequestering the activity of the biomolecules. These may be used, for example, to inhibit specific gene transcription in cancerous tissues.

Cancer is a major cause of morbidity and mortality despite our current best efforts at prevention and treatment. Cancer is caused by abnormal regulation of cellular growth processes including aberrations in the control of gene transcription. Research aimed at understanding the normal regulation of cell growth is crucial for future recognition and therapeutic modification of aberrant cell cycle regulation. For example, specific types of human papillomaviruses (HPVs) are closely associated with the development of cervical cancer. The transforming ability of these high-risk HPV types depends on the expression of the viral E6 and E7 oncogenes. It is therefore of particular interest to elucidate the molecular mechanisms that result in the activation of E6/E7 expression during HPV-associated tumorigenesis. Recently, much progress has been made in characterizing the proteins involved in the regulation of HPV oncogene transcription. Definition of the factors that regulate oncogene transcription is expected to provide new insights into the molecular mechanisms activating viral oncogene expression during carcinogenesis and forms an experimental basis for investigating the specific biochemical pathways that contribute to malignant cell transformation. Hoppe-Seyler, F., Butz, K., Mol. Carcinog., 10 (3):134 (1994).

Moreover, nucleic acids which encode the single chain monoclonal antibodies and fusion reagents identified by the methods described herein are contemplated for use in gene therapy for the control of congenital disease. See. e.g., Taneja, S. S., Pang, S., Cohan, P., Belldegrun, A., Gene Therapy: Principles and Potential, Cancer Surv., 23: 247 (1995).

Gene Therapy

Gene fusions of the present invention are incorporated into effective eukaryotic expression vectors, which are directly administered or introduced into somatic cells for gene therapy (mRNA transcripts of the gene fusion constructions may also be administered directly or introduced into somatic cells). Such vectors may remain episomal or may be incorporated into the host chromosomal DNA as a provirus or portion thereof that includes the gene fusion and appropriate eukaryotic transcription and translation signals, i.e, an effectively positioned RNA polymerase promoter 5′ to the transcriptional start site and ATG translation initiation codon of the gene fusion as well as termination codon(s) and transcript polyadenylation signals effectively positioned 3′ to the gene fusion.

The construction and use of retroviral vectors is well known to those of skill in this art (see, e.g., Eglitis, M. A., et al. Retroviral Vectors for Introduction of Genes into Mammalian Cells, Bio Techniques 6:608 (1988); Hodgson, C. P., et al. Retroviral Vectors for Gene Therapy and Transgenics, Curr. Opin. Ther. Patents, 3:223 (1993)). Advances in human gene therapy include the design of synthetic retrotransposon vectors, which may be used to practice the method of the present invention in humans (Chakrabarty, A. K., et al. FASEB Journal, 7:971 (1993).

Other advances in the development of retroviral vectors for human gene therapy include: Meyer, J., et al. Gene, 129:263 (1993); Matsushita, T. et al. Thrombosis Research, 69:387 (1993) (describes the construction of a new MoMLV-based retroviral vector for stable gene expression wherein 1.2 μg of gene product was produced per 10⁶ transformed cells/24hrs.); Chambers, C. A., et al., Proc. Natl. Acad. Sci., 89:1026 (1992). The replication defective retroviral vector derived from MoMuLV as described in Dranoff et al Proc. Natl. Acad. Sci., 90:3539 (1993) is particularly preferred to practice the method of the present invention.

Diagnostic use

Synthetic antibodies identified from screening methods described herein can be used for diagnostics including the identification of disease markers. The single chain monoclonal antibodies having affinity for transcription associated biomolecules are also useful for the diagnosis of pathological conditions as well as cancers manifested by overactive transcription of growth factors.

Diagnostic assays for transcription associated biomolecules include methods utilizing an antibody and a label to detect the transcriptional associated biomolecule population or bioconcentration in human body fluids, cells, tissues or sections or extracts of such tissues—as compared to the bioconcentration in normal tissue. The antibodies of the present invention may be used with or without modification. The antibodies may be labeled by joining them, either covalently or noncovalently, with a wide variety of well known different reporter molecules, preferably horseradish peroxidase.

A variety of protocols for measuring a transcriptional associated biomolecule, using the single chain monoclonal antibodies with affinity for transcription associated biomolecules are known in the art. Examples include enzyme-linked immunosorbent assay (ELISA), radioimmunoassay (RIA) and fluorescent activated cell sorting (FACS). A two-site, antibody-based immunoassay utilizing the single chain monoclonal antibodies-with affinity for transcription associated biomolecules reactive to two non-interfering epitopes on a transcriptional associated biomolecule is preferred, but a competitive binding assay may be employed. These assays are well known and are described, among other places, in Maddox, Del. et al (1983, J Exp Med 158:1211).

In order to provide a basis for the diagnosis of disease, normal or standard values for expression of the biomolecule of interest are established. This is accomplished by combining body fluids or cell extracts taken from normal subjects, either animal or human, with an antibody described herein under conditions suitable for complex formation which are well known in the art. The amount of standard complex formation can be quantified by comparing it with a dilution series of positive controls where a known amount of antibody is combined with known concentrations of the biomolecule of interest. Then, standard values obtained from normal samples may be compared with values obtained from samples from subjects potentially affected by a disorder or disease related to a chemokine receptor polypeptide expression. Deviation between standard and subject values establishes the presence of the disease state.

A method for diagnosing a physiological disorder manifested by abnormal levels of a transcription associated biomolecule is herein provided. The method comprises contacting a biological sample with a labelled single chain monoclonal antibody fusion reagent or a portion thereof whereby the antibody reagent binds to the transcription associated biomolecule to form a complex, and separating unbound labelled antibody reagent from the complex, measuring the amount of bound labelled antibody reagent in the complex; and, comparing the quantity of labelled antibody reagent in the biological sample to the quantity of labelled antibody reagent which binds to normal biological samples under identical conditions.

Drug Screening

Synthetic antibody libraries described herein can be used in any drug screening or ligand screening procedures. The synthetic antibodies identified from screening methods described herein can be used for screening a plurality of compounds for specific binding affinities to identify compounds associated with activating and inhibiting the expression of transcription associated biomolecules in a cell for the diagnosis, study, prevention and treatment of disease.

The present invention provides single chain monoclonal antibodies with affinity for transcription associated biomolecules as well as genetically engineered host cells that express the reagents to evaluate, screen and identify compounds, in appropriate cellular supernatants. The single chain monoclonal antibodies with affinity for transcription associated biomolecules of the present invention and genetically engineered host cells that express the reagents described herein may be used to help identify substances, compounds or synthetic drugs that modulate binding thereby modulating transcriptional activation. For example, the single chain monoclonal antibodies could be used to screen peptide libraries or organic molecules capable of modulating transcriptional activity.

In an embodiment of the present invention, single chain monoclonal antibodies with affinity for transcription associated biomolecules for instance that are demonstrated to neutralize the activity of a transcription associated biomolecule or variants thereof may be used to screen for peptides or other molecules, such as organic or inorganic molecules made by combinatorial chemistry; e.g. via affinity purification, that modulate transcriptional activity, to identify a therapeutic compound capable of modulating transcription.

A single chain monoclonal antibody with affinity for transcription associated biomolecules or oligopeptides thereof can be used for screening therapeutic compounds in any of a variety of drug screening techniques. The fragment employed in such a test may be free in solution, affixed to a solid support, displayed on a cell surface, or located intracellularly. The abolition of activity or the formation of binding complexes, between a reagent of the invention and the agent being tested, may be measured. Accordingly, the present invention provides a method for screening a plurality of compounds for specific binding affinity with the single chain monoclonal antibody with affinity for transcription associated biomolecules or a fragment thereof, comprising providing a plurality of compounds; combining a reagent of the present invention or a fragment thereof with each of a plurality of compounds for a time sufficient to allow binding under suitable conditions; and detecting binding of the reagent, or fragment thereof, to each of the plurality of compounds, thereby identifying the compounds which specifically bind the single chain monoclonal antibody with affinity for a transcription associated biomolecule. In such an assay, the plurality of compounds may be produced by combinatorial chemistry techniques known to those of skill in the art.

Another technique for drug screening provides for high throughput screening of compounds having suitable binding affinity to a single chain monoclonal antibody with affinity for a transcription associated biomolecule and is described in detail in Geysen, European Patent Application 84/03564, published on Sep. 13, 1984, which is incorporated herein by reference. In summary, large numbers of different small peptide test compounds are synthesized on a solid substrate, such as plastic pins or an alternate surface. The peptide test compounds are reacted with the reagent fragments and washed. A bound antibody of the present invention is then detected by methods well known in the art. A purified antibody reagent can also be coated directly onto plates for use in the aforementioned drug screening techniques. Alternatively, antibody reagents can be used to capture the biomolecule and immobilize it on a solid support.

Production of single chain monoclonal antibody fusion reagents

The selected variable regions of interest can be cloned into human or murine immunoglobulin expression vectors currently available to produce large amounts as desired. For example, expression vectors pSEC-Tag A, B and C (Invitrogen, San Diego, Calif.) have been successfully used. These particular vectors allow expression of single chain monoclonal antibody fusion reagents under the direction of the CMV promoter, and provide an immunoglobulin leader peptide for efficient secretion, and a myc epitope tag with which to evaluate expression of the sFv, as well as a poly-Histidine sequence at the C-terminus for simple purification on a nickel-chelating resin. Since sFv's have been found to be more rapidly cleared from the body of test animals and show more rapid tumor penetration, this expression system will allow researchers to produce biologically active and potentially pharmaceutically important molecules. These particular vectors are also useful to express fusion reagents of the present invention into media by tissue cultured cells, both transiently and stably and this expression can be monitored by virtue of the myc epitope tag.

The yeast expression vector pPICZ alpha B (Invitrogen, San Diego, Calif.) and the bacterial expression vector pNUT (discussed infra) are other example expression vectors which may be used to express the single chain fusion reagents described herein.

Transcription associated biomolecules

The CREB/ATF family of transcriptional regulatory biomolecules is used to exemplify screening for and isolation of single chain monoclonal antibody fusion reagents that specifically target transcription associated biomolecules. Specific fusion reagents are isolated that bring transcriptional activating peptides to individual members of the transcriptional regulatory biomolecules. A specific system is exemplified that targets constitutive transcriptional activation domains to endogenous signal-responsive transcriptional regulatory proteins. This technology allows the identification of diverse members of the family of biomolecules which are bound to regulatory sequences in vivo.

The novel screening method described herein can be used, for example, for the isolation of immunoglobulin regions that target hormonally-responsive transcription factors that are normally only active when they are stimulated in response to intracellular signaling pathways. Members of the CREB/ATF family of transcriptional regulatory proteins manifest this type of transcriptional regulation. Individual members share several characteristics including a bZIP domain involved in DNA-binding and dimerization. Hai, T, Liu, F, Coukos, W, Green, M. Genes Dev. 3:2083-2090, 1989; Hoeffler, JP, Meyer, T, Yun, Y, Jameson, J, Habener, JF. Science 257:680-682, 1988. Moreover, these proteins are defined by the DNA sequence to which they bind, which has the consensus 5′-TGACGTCA-3′. These factors exist in the nucleus bound to 5′ regulatory sequences of the genes which they influence. They are bound to DNA but are inactive until they are phosphorylated. Gonzalez, GA, et al., Nature 337:749-751, 1989; Lee, CQ, Yun, Y, Hoeffler, JP, Habener, JF. EMBO J. 9:4455-4465, 1990. Once phosphorylated an allosteric structural change exposes a transcriptional activating domain that previously existed in a masked configuration (some members of the CREB/ATF family of transcriptional regulatory proteins do not require phosphorylation).

Identification of transcriptional associated proteins which are bound to regulatory sequences in vivo is critical. For instance, as demonstrated by the CREB/ATF regulatory system, since in vitro assays of DNA-binding suggest that all members of the family will bind most if not all variants of the regulatory DNA sequence. The overexpression of the CREB/ATF proteins has the standard disadvantage of a large family of ubiquitously expressed endogenous proteins that all bind the same consensus motif in vitro. This makes interpretation of these types of experiments almost impossible.

Significance

Single chain monoclonal antibody fusion reagents of the present invention allow, for example, the determination of whether there is promiscuous binding of the different members of the CREB/ATF family to promoters in vivo, or whether there is specificity (Example VI).

CREB/P-BOX

An example method for isolating single chain monoclonal antibody fusion reagents that target constitutive transcriptional activation peptide domains to endogenous signal-responsive transcriptional regulatory proteins is an embodiment wherein the CREB phosphorylation BOX peptide domain (CREB/P-BOX) is fused to the LexA DBD and acts as the LexA DBD/protein antigen X fusion of interest (the “bait”) for screening immunoglobulin variable regions in this system. See FIG. 1.

When a single chain antibody fusion reagent molecule targets the CREB sequence in the antigen fusion, transcription factor function is reconstituted and the reporter genes are activated allowing growth on selective media lacking histidine, as well as demonstrating β-galactosidase (β-gal) activity. Positive interactions can be detected in this particular embodiment by selecting on plates lacking histidine, followed by a second screen for β-galactosidase expression. Identification of the immunoglobulin fusion reagent (antibody/VP16* fusion, for example) which binds the LexA DBD/CREB/P-BOX antigen fusion is the ultimate goal of the screening protocol. Moreover, once isolated, the nucleic acid sequences which encode the immunoglobulin fusion reagent can be cloned into a mammalian expression vector and the targeting of CREB in the nucleus may be ascertained using reporter genes and endogenous genes that are known to harbor consensus cAMP responsive element (CRE) motifs.

Construction of example bait strains

Two peptide antigen strains, ATF-2FL and CREBIP-BOX were constructed (see Example I) as LexA DBD fusions in pBTM116 (ATCC access #______), to screen the antibody fusion reagent library. The peptide antigen (X) “bait” strains were constructed using the pBTM116 yeast expression plasmid for example (FIG. 3) which contains a Trp1 gene for selection in yeast and the DBD of Lex A with a downstream polylinker to allow generation of Lex A DBD/antigen X (“bait”) fusion proteins. Vojtek, A. B., Hollenberg, S. M., Cooper, J. A., Cell, 74:205 (1993).

Construction of a yeast expression library vector (pVP16Zeo) with zeocin selection to facilitate the isolation of the antibody/VP16 fusion reagent plasmids

The yeast expression library vector pVP16Zeo (ATCC access #______) is constructed from three parent constructs, pPICZB (Invitrogen, San Diego), pGBT9 (Clonetech), and pVP16 (Vojtek, A. B., Hollenberg, S. M., Cooper, J. A., Cell, 74:205 (1993)), for the construction and screening of single chain monoclonal antibody fusion reagent libraries, comprising zeocin selection to facilitate the isolation and production of single chain monoclonal antibody fusion reagents. Selection in pVP16Zeo is based on a single selectable marker that confers resistance to the drug Zeocin in both Saccharomyces cerevisiae and E. coli. Collis,CM, Hall, RM. Plasmid 14:143-151, 1985; Wenzel, TJ, Migliazza, A, Ydesteensma, H, Vandenberg JA. Yeast 8:667-668, 1992. Zeocin selection is also compatible with either trp or leu selectable markers which may be used as “bait” plasmid markers. See Example VIII.

The ADH promoter as well as the ADH terminator and the 2 μm element is also included in pVP16zeo as shown in FIG. 5. The HindIII-EcoR1 fragment contains the ATG, NLS, SfiI-NotI sites for inserting the antibody library, a second NLS, VP16 transactivation domain and stop codons in all three reading frames. The second cassette contains the TEF1 yeast promoter, EM7 bacterial promoter, Zeocin resistance gene (sh/ble), cyc and f1 ori. This entire cassette can be isolated from an existing pPICZ Pichia pastoris vector (Invitrogen, San Diego Calif.) by digestion with BamHI and BgIII to generate a 1.9 kb fragment. BgIII linkers are added to the NarI/AatII cassette 1 so it can be combined with the BamHI/BgIII cassette 2 to create pVP16Zeo. The original stuffer fragment 1.5 kb ATF-2FL is cloned into the SfiI/NotlI sites of cassette 1. This serves two purposes, the first being visual verification of SfiI/Not1 digestion of pVP16Zeo by dropping out the 1.5 kb ATF-2FL piece when the sFv library is cloned in. The second purpose is since the stuffer is an ATF-2FL/VP16 fusion the function of the library vector may be tested before cloning in a new library by transforming the pVP16Zeo (with the ATF-2FL stuffer fragment) into the antigen bait strain ATF-2FL/BTM116. ATF-2FL/VP16 fusions will dimerize with the ATF-2FL/LexA fusion in the bait strain and produce a positive interaction that will be detected by both growth on plates lacking histidine and blue color in a β-gal assay.

Human sFv library

Generation of cDNA: PCR Amplification Total RNA is isolated for example from 3 human peripheral blood lymphocyte preparations (San Diego Blood Bank, Calif.) and portions of 4 human spleens using a SNAP™ Total RNA kit (Invitrogen, San Diego). The pooled total RNA is used in four separate first strand cDNA synthesis reactions and primed with one of the four constant region-specific primers (SEQ ID NO: 3-6). See Table I. These four oligonucleotides are specific to either the human heavy chain, IgM and IgG, or light chain, lambda and kappa, constant regions. The first strand reactions are performed using a cDNA Cycle Kit (Invitrogen) and subjected to two consecutive rounds of transcription. The heavy and light chain variable genes are PCR amplified from the cDNA using a mixture of family-specific human V-gene back primers and human germ-line J-segment forward primers (SEQ ID NO: 7-86). See Table I-III. The product from each PCR is run on a 1% agarose gel and purified using Geneclean, Bio 101, and then reamplified with similar primers containing restriction sites. See Tables I-III. These primer pairs add an ApaLI and NotI site to the light chain segments or an SfiL and Sall site to the heavy chain fragments.

TABLE I
HUMAN V GENE PRIMER

SEQ ID NOs: 3-4 Human heavy chain constant region primer

| | | | | | | | | | | | |
|---|---|---|---|---|---|---|---|---|---|---|---|
| 3 | Ig M | 5′ | TGG | AAG | AGG | CAC | GTT | CTT | TTC | TTT | 3′ |
| 4 | IgG CH for | 5′ | GTC | CAC | CTT | GGT | GTT | GCT | GGG | CTT | 3′ |

SEQ ID NOs: 5-6 Human light chain constant region primer

| | | | | | | | | | | | | |
|---|---|---|---|---|---|---|---|---|---|---|---|---|
| 5 | Ck for | 5′ | AGA | CTC | TCC | CCT | GTT | GAA | GCT | CTT | 3′ | |
| 6 | C1 for | 5′ | TGA | AGA | TTC | TGT | AGG | GGC | CAC | TGT | CTT | 3′ |

SEQ ID NOs: 7-28 Human VH primer
Initial amplification primer

| | | | | | | | | | | | |
|---|---|---|---|---|---|---|---|---|---|---|---|
| 7 | VH1a back | 5′ | CAG | GTG | CAG | CTG | GTG | CAG | TCT | GG | 3′ |
| 8 | VH1b back | 5′ | CAG | GTG | CAG | CTG | GTG | GAG | TCT | GG | 3′ |
| 9 | VH1c back | 5′ | CAG | GTC | CAG | CTT | GTG | CAG | TCT | GG | 3′ |
| 10 | VH2a back | 5′ | CAG | GTC | ACC | TTG | AAG | GAG | TCT | GG | 3′ |
| 11 | VH2b back | 5′ | CAG | ATC | ACC | TTG | AAG | GAG | TCT | GG | 3′ |
| 12 | H3 back | 5′ | GAG | GTG | CAG | CTG | GTG | GAG | TCT | GG | 3′ |
| 13 | VH4 back | 5′ | CAG | GTG | CAG | CTG | CAG | GAG | TCG | GG | 3′ |
| 14 | VH5a back | 5′ | GAG | GTG | CAG | CTG | TTG | CAG | TCT | GC | 3′ |
| 15 | VH5b back | 5′ | GAG | GTG | CAG | CTG | GTG | CAG | TCT | GG | 3′ |
| 16 | VH5c back | 5′ | GAG | GTG | CAG | CTG | TTG | GAG | TCT | GG | 3′ |
| 17 | VH6 back | 5′ | CAG | GTA | CAG | CTG | CAG | CAG | TCA | GG | 3′ |

Reamplification primer with SfiI/NcoI appended restriction sites

| | | | | | | | | | | | | | | | | | | | | | | |
|---|---|---|---|---|---|---|---|---|---|---|---|---|---|---|---|---|---|---|---|---|---|---|
| 18 | VH1a ba SfiI | 5′ | GTC | CTC | GCA | ACT | GCG | GCC | CAG | CCG | GCC | ATG | GCC | CAG | GTG | CAG | CTG | GTG | CAG | TCT | GG | 3′ |
| 19 | VH1b ba SfiI | 5′ | GTC | CTC | GCA | ACT | GCG | GCC | CAG | CCG | GCC | ATG | GCC | CAG | GTG | CAG | CTG | GTG | GAG | TCT | GG | 3′ |
| 20 | VH1c ba SfiI | 5′ | GTC | CTC | GCA | ACT | GCG | GCC | CAG | CCG | GCC | ATG | GCC | CAG | GTC | CAG | CTT | GTG | CAG | TCT | GG | 3′ |
| 21 | VH2a ba SfiI | 5′ | GTC | CTC | GCA | ACT | GCG | GCC | CAG | CCG | GCC | ATG | GCC | CAG | GTC | ACC | TTG | AAG | GAG | TCT | GG | 3′ |
| 22 | VH2b ba SfiI | 5′ | GTC | CTC | GCA | ACT | GCG | GCC | CAG | CCG | GCC | ATG | GCC | CAG | ATC | ACC | TTG | AAG | GAG | TCT | GG | 3′ |
| 23 | VH3 ba SfiI | 5′ | GTC | CTC | GCA | ACT | GCG | GCC | CAG | CCG | GCC | ATG | GCC | GAG | GTG | CAG | CTG | GTG | GAG | TCT | GG | 3′ |
| 24 | VH4 ba SfiI | 5′ | GTC | CTC | GCA | ACT | GCG | GCC | CAG | CCG | GCC | ATG | GCC | CAG | GTG | CAG | CTG | CAG | GAG | TCG | GG | 3′ |
| 25 | VH5a ba SfiI | 5′ | GTC | CTC | GCA | ACT | GCG | GCC | CAG | CCG | GCC | ATG | GCC | GAG | GTG | CAG | CTG | TTG | CAG | TCT | GC | 3′ |
| 26 | VH5b ba SfiI | 5′ | GTC | CTC | GCA | ACT | GCG | GCC | CAG | CCG | GCC | ATG | GCC | GAG | GTG | CAG | CTG | GTG | CAG | TCT | GG | 3′ |
| 27 | VH5c ba SfiI | 5′ | GTC | CTC | GCA | ACT | GCG | GCC | CAG | CCG | GCC | ATG | GCC | GAG | GTG | CAG | CTG | TTG | GAG | TCT | GG | 3′ |
| 28 | VH6 ba SfiI | 5′ | GTC | CTC | GCA | ACT | GCG | GCC | CAG | CCG | GCC | ATG | GCC | CAG | GTA | CAG | CTG | CAG | CAG | TCA | GG | 3′ |

SEQ ID NOs: 29-40 Human JH primer
Initial amplification primer

| | | | | | | | | | | | |
|---|---|---|---|---|---|---|---|---|---|---|---|
| 29 | JH1 for | 5′ | TGA | GGA | GAC | GGT | GAC | CAG | GGT | GCC | 3′ |
| 30 | JH2 for | 5′ | TGA | GGA | GAC | AGT | GAC | CAG | GGT | GCC | 3′ |
| 31 | JH3 for | 5′ | TGA | AGA | GAC | GGT | GAC | CAT | TGT | CCC | 3′ |
| 32 | JH4 for | 5′ | TGA | GGA | GAC | GGT | GAC | CAG | GGT | CCC | 3′ |
| 33 | JH5 for | 5′ | TGA | GGA | GAC | GGT | GAC | CAG | GGT | TCC | 3′ |
| 34 | JH6 for | 5′ | TGA | GGA | GAC | GGT | GAC | CGT | GGT | CCC | 3′ |

Reamplification primer with SalI appended restriction site

| | | | | | | | | | | | | | | | |
|---|---|---|---|---|---|---|---|---|---|---|---|---|---|---|---|
| 35 | JH1 for SalI | 5′ | GAG | TCA | TTC | TCG | TGT | CGA | CAC | GGT | GAC | CAG | GGT | GCC | 3′ |
| 36 | JH2 for SalI | 5′ | GAG | TCA | TTC | TCG | TGT | CGA | CAC | AGT | GAC | CAG | GGT | GCC | 3′ |
| 37 | JH3 for SalI | 5′ | GAG | TCA | TTC | TCG | TGT | CGA | CAC | GGT | GAC | CAT | TGT | CCC | 3′ |
| 38 | JH4 for SalI | 5′ | GAG | TCA | TTC | TCG | TGT | CGA | CAC | GGT | GAC | CAG | GGT | CCC | 3′ |
| 39 | JH5 for SalI | 5′ | GAG | TCA | TTC | TCG | TGT | CGA | CAC | GGT | GAC | CAG | GGT | TCC | 3′ |
| 40 | JH6 for SalI | 5′ | GAG | TCA | TTC | TCG | TGT | CGA | CAC | GGT | GAC | CGT | GGT | CCC | 3′ |

TABLE II
SEQ ID NOs: 41-54 Human Vx primer
Initial amplification primer

| | | | | | | | | | | | |
|---|---|---|---|---|---|---|---|---|---|---|---|
| 41 | Vx1 back | 5′ | GAC | ATC | CAG | ATG | ACC | CAG | TCT | CC | 3′ |
| 42 | Vx2 back | 5′ | GAT | ATT | GTG | ATG | ACC | CAG | A/TCT | CC | 3′ |
| 43 | Vx3 back | 5′ | GAA | ATT | GTG | C/TTG | ACA/T | CAG | TCT | CC | 3′ |
| 44 | Vx4 back | 5′ | GAC | ATC | GTG | ATG | ACC | CAG | TCT | CC | 3′ |
| 45 | Vx5 back | 5′ | GAA | ACG | ACA | CTC | ACG | CAG | TCT | CC | 3′ |
| 46 | Vx6 back | 5′ | GAG | ATT | GTG | ATG | ACC | CAG | ACT | CC | 3′ |
| 47 | Vx10 back | 5′ | GAC | CAC | GTG | ATG | ACC | CAG | TCT | CC | 3′ |

Reamplification primers with ApoL 1 appended restriction sites

| | | | | | | | | | | | | | | | | | |
|---|---|---|---|---|---|---|---|---|---|---|---|---|---|---|---|---|---|
| 48 | Vx1 back ApoL 1 | 5′ | TGA | GCA | CAC | AGT | GCA | CTC | GAC | ATC | CAG | ATG | ACC | CAG | TCT | CC | 3′ |
| 49 | Vx2 back ApoL 1 | 5′ | TGA | GCA | CAC | AGT | GCA | CTC | GAT | ATT | GTG | ATG | ACC | CAG | A/TCT | CC | 3′ |
| 50 | Vx3 back ApoL 1 | 5′ | TGA | GCA | CAC | AGT | GCA | CTC | GAA | ATT | GTG | C/TTG | ACA/T | CAG | TCT | CC | 3′ |
| 51 | Vx4 back ApoL 1 | 5′ | TGA | GCA | CAC | AGT | GCA | CTC | GAC | ATC | GTG | ATG | ACC | CAG | TCT | CC | 3′ |
| 52 | Vx5 back ApoL 1 | 5′ | TGA | GCA | CAC | AGT | GCA | CTC | GAA | ACG | ACA | CTC | ACG | CAG | TCT | CC | 3′ |
| 53 | Vx6 back ApoL 1 | 5′ | TGA | GCA | CAC | AGT | GCA | CTC | GAG | ATT | GTG | ATG | ACC | CAG | ACT | CC | 3′ |
| 54 | Vx10 back ApoL 1 | 5′ | TGA | GCA | CAC | AGT | GCA | CTC | GAC | CAC | GTG | ATG | ACC | CAG | TCT | CC | 3′ |

SEQ ID NOs: 55-60 Human Jx primer
Initial amplification primer

| | | | | | | | | | | | |
|---|---|---|---|---|---|---|---|---|---|---|---|
| 55 | Jx1 for | 5′ | ACG | TTT | GAT | C/TTC | CAC/G | CTT | GGT | CCC | 3′ |
| 56 | Jx3 for | 5′ | ACG | TTT | GAT | ATC | CAC | TTT | GGT | CCC | 3′ |
| 57 | Jx5 for | 5′ | ACG | TTT | AAT | CTC | CAG | TCG | TGT | CCC | 3′ |

Reamplification primers with NotI appended restriction sites

| | | | | | | | | | | | | | | | | | | | |
|---|---|---|---|---|---|---|---|---|---|---|---|---|---|---|---|---|---|---|---|
| 58 | Jx1 for NotI | 5′ | GAG | TCA | TTC | TCG | ACT | TGC | GGC | CGC | ACG | TTT | GAT | C/TTC | CAC/G | CTT | GGT | CCC | 3′ |
| 59 | Jx3 for NotI | 5′ | GAG | TCA | TTC | TCG | ACT | TGC | GGC | CGC | ACG | TTT | GAT | ATC | CAC | TTT | GGT | CCC | 3′ |
| 60 | Jx5 for NotI | 5′ | GAG | TCA | TTC | TCG | ACT | TGC | GGC | CGC | ACG | TTT | AAT | CTC | CAG | TCG | TGT | CCC | 3′ |

TABLE III
SEQ ID NOs: 61-78 Human Vλ primer
Initial amplification primer

| | | | | | | | | | | | |
|---|---|---|---|---|---|---|---|---|---|---|---|
| 61 | Vλ1a back | 5′ | CAG | TCT | GTG | T/CTG | ACT/G | CAG | CCG | CC | 3′ |
| 62 | Vλ1b back | 5′ | CTG | TGC | TGA | CG/TC | AGC | CA/GC | CCT | CA | 3′ |
| 63 | Vλ2 back | 5′ | CAG | TCT | GCC | CTG | ACT | CAG | CCT | GC | 3′ |
| 64 | Vλ3a back | 5′ | CAG | ACT | GTG | GTG | ACC | CAG | GAG | CC | 3′ |
| 65 | Vλ3b back | 5′ | TCC | TCT | GAG | CTG | AGT | CAG | CAG | CC | 3′ |
| 66 | Vλ7 back | 5′ | CAG | GCT | GTG | GTG | ACT | CAG | GAG | CC | 3′ |
| 67 | Vλ8 back | 5′ | CTG | TGG | TGA | CCC | AGG | AGC | CAT | CA | 3′ |
| 68 | Vλ9 back | 5′ | CAG | CCT | GTG | CTG | ACT | CAG | CCA | CC | 3′ |
| 69 | Vλ10 back | 5′ | CCT | ATG | AGC | TGA | CTC | AGC | CAC | TC | 3′ |

Reamplification primers with ApoL 1 appended restriction sites

| | | | | | | | | | | | | | | | | | |
|---|---|---|---|---|---|---|---|---|---|---|---|---|---|---|---|---|---|
| 70 | Vλ1a back ApoL 1 | 5′ | TGA | GCA | CAC | AGT | GCA | CTC | CAG | TCT | GTG | T/CTG | ACT/G | CAG | CCG | CC | 3′ |
| 71 | Vλ1b back ApoL 1 | 5′ | TGA | GCA | CAC | AGT | GCA | CTC | CTG | TGC | TGA | CG/TC | AGC | CA/GC | CCT | CA | 3′ |
| 72 | Vλ2 back ApoL 1 | 5′ | TGA | GCA | CAC | AGT | GCA | CTC | CAG | TCT | GCC | CTG | ACT | CAG | CCT | GC | 3′ |
| 73 | Vλ3a back ApoL 1 | 5′ | TGA | GCA | CAC | AGT | GCA | CTC | CAG | ACT | GTG | GTG | ACC | CAG | CAG | CC | 3′ |
| 74 | Vλ3b back ApoL 1 | 5′ | TGA | GCA | CAC | AGT | GCA | CTC | TCC | TCT | GAG | CTG | AGT | CAG | CAG | CC | 3′ |
| 75 | Vλ7 back ApoL 1 | 5′ | TGA | GCA | CAC | AGT | GCA | CTC | CAG | GCT | GTG | GTG | ACT | CAG | GAG | CC | 3′ |
| 76 | Vλ8 back ApoL 1 | 5′ | TGA | GCA | CAC | AGT | GCA | CTC | CTG | TGG | TGA | CCC | AGG | AGC | CAT | CA | 3′ |
| 77 | Vλ9 back ApoL 1 | 5′ | TGA | GCA | CAC | AGT | GCA | CTC | CAG | CCT | GTG | CTG | ACT | CAG | CCA | CC | 3′ |
| 78 | Vλ10 back ApoL 1 | 5′ | TGA | GCA | CAC | AGT | GCA | CTC | CCT | ATG | AGC | TGA | CTC | AGC | CAC | TC | 3′ |

SEQ ID NOs: 79-86 Human Jλ primer
Initial amplification primer

| | | | | | | | | | | | |
|---|---|---|---|---|---|---|---|---|---|---|---|
| 79 | Jλ1 for | 5′ | ACC | TAG | GAC | CGT | GAC | CTT | GGT | CCC | 3′ |
| 80 | Jλ2 for | 5′ | ACC | TAG | GAC | GGT | CAG | CTT/C | GGT | CCC | 3+ |
| 81 | Jλ4 for | 5′ | ACC | TAA | AAT | GAT | CAG | CTG | GGT | TCC | 3′ |
| 82 | Jλ6 for | 5′ | ACC | GAG | GAC | GGT | CAC | CTT | GGT | GGC | 3′ |

Reamplification primers with NotI appended restriction sites

| | | | | | | | | | | | | | | | | | | | |
|---|---|---|---|---|---|---|---|---|---|---|---|---|---|---|---|---|---|---|---|
| 83 | Jλ1 for NotI | 5′ | GAG | TCA | TTC | TCG | ACT | TGC | GGC | CGC | ACC | TAG | GAC | GGT | GAC | CTT/C | GGT | CCC | 3′ |
| 84 | Jλ2 for NotI | 5′ | GAG | TCA | TTC | TCG | ACT | TGC | GGC | CGC | ACC | TAG | GAC | GGT | CAG | CTT | GGT | CCC | 3′ |
| 85 | Jλ4 for NotI | 5′ | GAG | TCA | TTC | TCG | ACT | TGC | GGC | CGC | ACC | TAA | AAT | GAT | CAG | CTG | GGT | TCC | 3′ |
| 86 | Jλ6 for NotI | 5′ | GAG | TCA | TTC | TCG | ACT | TGC | GGC | CGC | ACC | GAG | GAC | GGT | CAC | CTT | GGT | GGC | 3′ |

Cloning of sFv Fragments

The vector and light chain PCR products are first digested with ApaLI and NotI. After gel-purifying the vector backbone and light chain fragments, the two segments are ligated using T4 ligase. The library is transformed into Top10F cells, plated on LB-Ampicillin plates, and individual colonies screened for insertion of the light chain segments. The vector containing the light chain library, and the heavy chain PCR product are digested with SfiL and SailI and gel-purified. The light chain and heavy chain segments are randomly combined by ligating the heavy chain fragment into the vector containing the light chain fragments.

Human sFv library amplification and subcloning into a yeast expression vector

The isolation of single chain monoclonal antibody fusion reagents capable of targeting specific transcription factors in vivo is performed for example by cloning a library of human-derived monoclonal antibody variable domains (sFv's) into a yeast expression vector that encodes fusion proteins linking these sFv's with nuclear localization signals and a constitutive transactivation domain (VP16).

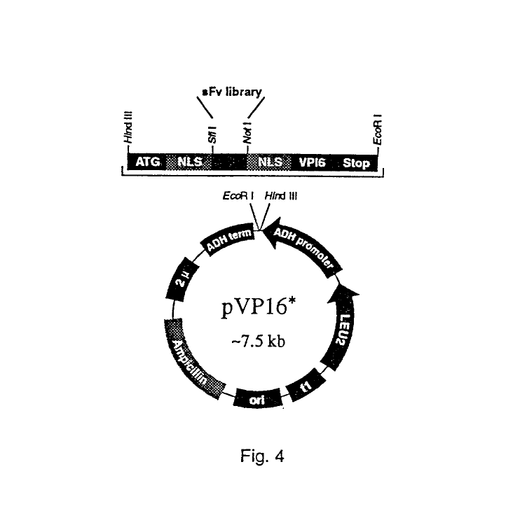

In this example a library of single chain antibody molecules was cloned into an expression plasmid PVP16* (FIG. 4) between a 5′ nuclear localization signal and a 3′ nuclear localization signal and the VP16 transactivation domain. Marks, JD, Hoogenboom, HR, Bonnert, TP, McCafferty, J, Griffiths, AD, Winter, G. Mol. Biol. 222:581-597, 1991. The yeast expression plasmid (pVP16*) (FIG. 4) was constructed to express a library of human sFv's as fusion proteins with a duplicate nuclear localization sequence (NLS) derived from the SV40 T Ag, where the sFv is to be positioned between, and the VP16 acidic activation domain at the C-terminus. The parent plasmid pVP16 was modified by inserting a double stranded oligonucleotide with XhoI compatible overhangs into the XhoI site 5′ of the NLS. Vojtek, A. B., Hollenberg, S. M., Cooper, J. A., Cell, 74:205 (1993). The synthetic oligonucleotide inserted contained an ATG followed by a NLS and recognition sequences for Sfil and NotI that were compatible for insertion of sFv sequences in frame with the second NLS and VP16. Biocca, S., et al., Trends in Cell Biology, 5:248 (1995); Kalderon, D., et al., Cell, 39:499 (1984); Biocca, S., et al., Biotechnology, 13:1110(1995).

To check for digestion of pVP16 at SfiI and NotI for insertion of the sFv library, a ATF-2FL stuffer fragment was cloned into these sites. Maekawa, T., et al., EMBO J., 8:2023 (1989). ATF-2FL was PCR amplified via standard procedures to add a 5′ SfiI site and a 3′ NotI site, with no stop codon, so a fusion with VP16 would be produced. This allows an agarose gel-visible 1.5 kb fragment to drop out when pVP16* is digested with SfiI and NotI.

| | |
|---|---|
| Primers: | |
| 5′ (Sfi1/ATF2) AGTGGCCCAGCCGGCCAAATTCAAGTTACATGTGAATT 3′ | (SEQ ID NO:87) |
| 5′ (Not1/ATF2) GAGGCGGCCGCACTTCCTGAGGGCTGTGACTGGG 3′ | (SEQ ID NO:88) |

Cotransformation of an ATF-2FL/Lex A DBD/BTM116 peptide antigen for selection (bait) plasmid and the ATF-2FL/VP16* immunoglobulin variable region for screening plasmid results in activation of the reporter construct (e.g. His 3 or Lac Z) because ATF-2FL will dimerize in the nucleus and thus bring the VP16.TA to the Lex A DBD.

These two constructs were cotransformed in yeast and grew on His− plates with a strong β-galactosidase activity shown with a filter assay proving that the library vector pVP16* was functional. See Examples III and IV. The Lex A bait strains alone had no background growth on plates lacking histidine.

Construction and characterization of the sFv library in pVP16*

The sFv library was shuttled into pVP16* by digesting a preexisting human sFv library with SfiI and NotI and isolating a band of approximately 0.8 kb sFv fragments to be inserted on a low melt agarose gel. Marks, JD, Hoogenboom, HR, Bonnert, TP, McCafferty, J, Griffiths, AD, Winter, G. Mol. Biol. 222:581-597, 1991. The pVP16* vector was also digested with SfiI and NotI and the resulting linear vector was isolated from the ATF-2FL stuffer fragment. The sFv inserts were ligated into the SfiI/Notl linearized pVP16* and transformed by electroporation into electrocompetent INVαF bacteria (Invitrogen, San Diego, Calif.). Two separate ligations were performed and combined for a final library size of 3.6×10⁶. See Example II. The 3.6×10⁶ member sFv library was reasonably diverse as verified by fingerprinting amplified clones by comparison of BstN I restriction fragment sizes. PCR amplification of 24 resulting clones was performed using standard procedures; 22 of the picked clones had insert. BstNI digests were also performed on the chosen PCR products to check diversity; there were at least 13 different digest patterns of the 24 tested. Library stocks were frozen at −20° C. as 20% glycerol stocks. Aliquots of the library stock were subsequently used to prepare DNA for large scale transformations.

Preparation of Expression Library

Subclone a human sFv library into pVP16Zeo to produce a human sFv expression library in yeast

An antibody library is shuttled into pVP16Zeo for instance by digesting a preexisting human sFv library with Sfil and NotI and isolating −0.8 kb sFv fragments on a low melt agarose gel as described supra. Marks, JD, Hoogenboom, HR, Bonnert, TP, McCafferty, J, Griffiths, AD, Winter, G. Mol. Biol. 222:581-597, 1991. The pVP16Zeo is also digested with SfiI and NotI and the cut vector isolated from the 1.5 kb ATF-2 stuffer fragment. The sFv inserts and cut pVP16Zeo are ligated (with optimized ratios of insert to vector) and transformed by electroporation into electrocompetent INVaF′ bacteria (Invitrogen, San Diego, Calif.) for example. A library of transformants of at least 10⁶−10⁷ individual recombinants should be obtained for good diversity. PCR amplification of representative clones is done to verify the presence of insert. BstNI digests are performed on the PCR products to check diversity of the new library. Library stocks are frozen as 20% glycerol stocks and aliquots used to prepare library DNA for large scale transformations. The diversity of the library doesn't need to be much above 10⁶ since the transformation capacity of yeast is generally 10⁷ or below.

Screen the human sFv library in yeast to isolate molecules that target CREB and ATF-2, and test their specificity in this system

Multiple sFv clones were isolated in both LacZ and His3 screens. See Example IV.

The first library was screened with the ATF-2FL/BTM116 bait strain. As shown (Flowchart (I)) approximately 4×10⁶ Trp⁺ Leu⁺ transformants were screened and 121 His⁺ clones were seledted. Of the 121 His⁺ clots also β-Gal positive.

Flowchart (I)

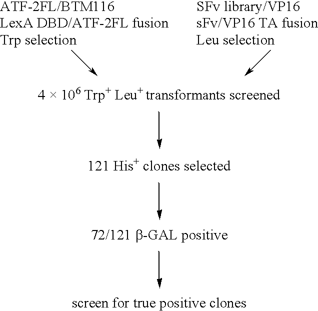

| β-GAL activity | positive | negative |
|---|---|---|
| ATF-2FLsFv/VP16 | | X |
| ATF-2FLsFv/VP16 + | | X |
| BTM116 | | |
| ATF-2FLsFv/VP16 + | | X |
| lamin/BTM116 | | |
| ATF-2FLsFv/VP16 + | X | |
| ATF-2FL/BTM116 | | |
| | 7 true positive clones | |

Tell the targeting specivicitly of the isolated clones and characterize in vitro with bacterially expressed sFv's as reagents on western blots and in vivo by expression of the sFv's in mammalian cells lots and in vivo by expression of the sfv's in mammalian cells

Expression of both sets of sFv clones has been accomplished in bacteria. See Example V. These clones were demonstrated to be specific and capable of recognizing their appropriate antigens in vitro as determined by using periplasmic preparations of the sFv's as primary antibodies on western blots (See Examples VI and VII).

Screening a pVP16Zeo expression library with a variety of antigens

Now that it has been shown that this technology can be used to isolate sFv's in vivo, s useful reagents may be isolated for studying a diverse range of signal transduction pathways and for controlling transcription. The CREB/ATF factor family is one embodiment to create a panel of reagents that will preferably exhibit specificity between different family members in vivo. Another family of preferred antigenic baits within the scope of the invention comprises various intracellular signaling molecules including small GTP binding proteins (Ras), adaptor proteins (Grb2), second messengers (phospholipase Cγ-PLCγ, phosphatidylinositol 3-kinase-PI3K), protein phosphatases (Syp) and kinases (mitogen activated protein kinase-MAPK, jun kinase-JNK). A third preferred family of contemplated antigenic baits are the nuclear hormone receptors including the androgen receptor (AR), thyroid hormone receptor (TR) and the glucocorticoid receptor (GR). These embodiments comprise a wide panel of useful targeting reagents that can be used in conjunction with specific sFv targeting vectors.

Antigens including multiple members of the CREB/ATF transcription factor family (ATF-1, ATF-3, ATF-4, ATF-6, CREMτ), intracellular signaling molecules (Ras, Grb2, PLCγ, PI3.K, Syp, MAPK, JNK), and DNA binding domains of nuclear hormone receptors (including but not limited to androgen receptor, thyroid hormone receptor, glucocorticoid receptor) are contemplated bait antigens to be used for screening single chain monoclonal antibody fusion reagents.

The CREB/ATF family of transcription factors, all contemplated as bait antigens for use in the current invention, share a conserved basic region/leucine zipper (bZIP) motif which is involved in DNA binding, but diverge in other regions. It has become clear that each member plays both different and overlapping roles in signal transduction pathways. ATF-1 is similar to CREB in that it stimulates transcription in response to cAMP. ATF-3 is induced by many physiological stresses including both mechanically and chemically induced. Liang, G, Wolfgang, CD, Chen, BPC, Chen, T-H, Hai, T. J. Biol. Chem. 4:1-7, 1996. ATF-4 and ATF-6 are two additional members of the family; ATF-4 is known to interact with distinct jun/fos proteins. CREMτ (cAMP responsive element modulator) is an activator of transcription (other CREM isoforms are repressors which are also contemplated as bait antigens for use in the current invention). Both full length proteins and crucial regions (such as the phosphorylation box, bZIP domain) are contemplated embodiments.

Hormonal activation of signal transduction pathways links extracellular signals to intracellular signals commonly referred to as second messengers which eventually influence transcriptional responses resulting in the activation of many cellular genes. Malarkey, K, Belham, CM, Paul, A, Grahm, A, McLees, A, Scott, PH, Plevin, R. Biochem J. 309:361-375, 1995. In this way hormones are able to regulate processes as diverse as homeostasis, reproduction, development, differentiation, mitogenesis and oncogenesis. Transcriptional control of eukaryotic gene expression is tightly regulated by the binding of nuclear factors to control elements. The availability of these factors is determined by cell type, differentiation state and position in the cell cycle. The identification and characterization of numerous cellular signaling proteins has progressed rapidly because of technology enabling the introduction of expression plasmids into mammalian cells. The subsequent characterization of the effect (on cellular growth and differentiation) of constitutively expressing an otherwise tightly regulated molecule has permitted the elucidation of many complex signaling pathways.

Specific targeting antibodies against signaling molecules are valuable research tools to help characterize the function of these proteins in vivo. Toward this end adaptor proteins (Grb2), second messengers (PLCγ, P13K), kinases (MAPK, JNK), phosphatases (Syp) and the small GTP binding protein Ras may be targeted as the antigen bait using the methods described herein. The cDNAs and corresponding expression constructs of all of these signaling molecules are currently available for construction of the antigen bait strain source of the proteins for selection and characterization of single chain monoclonal antibody fusion reagents.

The DNA binding domain (DBD) of the androgen receptor (AR) encoded by amino acids 559-624 contains two zinc fingers (1-residues 559-579 and 2-residues 595-619). Quigley, CA, De Bellis, A, Marschke, KB, El-awady, MK, Wilson, EM, French, FS. Endo. Rev. 16:271-321, 1995. A portion of the DNA binding region interacts with transcriptional enhancer nucleotide sequences referred to as HREs (hormone response element) and regulates target gene expression. Using this portion of the AR as an antigen bait for screening the antibody fusion reagent library is expected to yield preferred reagents that inhibit AR regulation of target genes in vivo. In particular the negative regulation of lutenizing hormone (both the α and β subunit genes) by androgens are expected to be blocked by anti-ARs antibodies. Clay, CM, Keri, RA, Finicle, AB, Heckart, LL, Hamernik, DL, Marschke, KM, Wilson, EM, French, FS, Nilson, JH. J. Biol. Chem. 268:13556-13564, 1993. Other embodiments within the scope of the invention include members of the nuclear hormone receptor family and the thyroid hormone receptor and the glucocorticoid receptor which are also contemplated to be bait antigens for antibody selection.

Transgenic Expression of the Fusion Reagents

Typically elements required for transcription of specific genes are identified using cell culture paradigms. The trans-acting factors that interact with these elements are then identified using nuclear extracts from cell lines or from complex tissues containing multiple cell types. Transgenic expression of single chain monoclonal antibody fusion reagents of the present invention may be used to identify as well as control specific elements required for the transcription of specific genes.

The single chain monoclonal antibodies of the present invention are contemplated to be expressed in transgenic animals in order to provide systems to mimic and study human diseases. In particular transgenic expression is to enable the determination of the specificity of transcription factor binding in vivo. Transgenic animals will allow the determination of putative transcriptional regulatory protein endogenous binding and regulation of specific promoters in vivo.

The glycoprotein α-subunit promoter, for instance, has been shown using cell culture paradigms, to contain a tandem cAMP response element (CRE) that binds members of the CREB and other members of the bZIP family of DNA binding proteins. Lee, CQ, Yun, Y, Hoeffler, JP, Habener, JF. EMBO J. 9:4455-4465, 1990; Heckert, LL, Schultz, K, Nilson, JH. J. Biol. Chem. 270:26497-26504, 1995; Jamenson, JL, Hollenberg, AA. Endocrine Rev. 14:203-221, 1993. In addition, cAMP and CREB play a major role in somatotrope homeostasis. However, CREB deficient mice lack conspicuous pituitary pathology. Hummler, E, Cole, TJ, Blendy, JA, Ganss, R, Aguzzi Schmid, A, Beerman, F, Schutz, G. PNAS 91:5847-5851, 1994. This suggests that multiple factors mediate the regulatory effects of the CRE. Expression of dominant negative single chain antibodies should inactivate, for instance, all functional CREB or ATF-2 in the cells that have been targeted and should allow assessment of the role of each of these factors in regulating transcription of the GH and α-subunit genes. Moreover, expression of antibody/VP16 activation domain fusions should produce constitutively active CREB and ATF-2 molecules in targeted cells.

To address the role of CREB and transcription factor, ATF-2, in regulating these native genes, specific constructs to target expression of antibody fusion reagents directed against these two factors to somatotropes and gonadotropes in vivo are contemplated as example embodiments. Lee, CQ, Yun, Y, Hoeffler, JP, Habener, JF. EMBO J. 9:4455-4465, 1990. Two cell types in the murine pituitary: gonadotropes that express the glycoprotein hormone α, LHβ, and FSHβ genes; and somatotropes that express growth hormone are targets for this example of the present invention applied in transgenic mice.

To target gonadotropes, the bovine α promoter is used which directs high level gonadotrope-specific expression of multiple reporter genes including chloramphenicol acetyl transferase (CAT), HSV thymidine kinase (unpublished data), β-galactosidase (unpublished data), diphtheria toxin, and a novel form of the LHβ subunit in transgenic mice. To target somatotropes, the rat growth hormone (GH) promoter is used. This promoter has been shown to yield high level expression of reporter genes encoding growth hormone (Lira, SA, Crenshaw, EBI, Glass, CK, Swanson, LW, Rosenfeld, MG. PNAS 85:4755-4759, 1988), HSV thymidine kinase (Borrelli, E, Heyman, RA, Arias, D, Sawchenko, PE, Evans, RM. Nature 339:538-541, 1989) diphtheria toxin (Behringer, RR, Mathews, LS, Palmiter, RD, Brinster, RL. Genes & Devel. 2:453-461, 1988), cholera toxin (Burton, FH, Hasel, KW, Bloom, FE, Sutcliffe, JG. Nature 350:74-77, 1991), and a dominant negative form of CREB (Struthers, RS, Vale, WW, Arias, C, Sawchenko, PE, Montminy, MR. Nature 350:622-624, 1991) solely to somatotropes in transgenic mice. A dominant negative CREB molecule has previously been expressed in transgenic mice using the GH promoter. These mice had the obvious phenotype of dwarfism. Thus, the GH-antibody fusion constructs of the present invention are expected to function properly. Both promoters are linked 5′ to the coding sequences of the various single chain antibodies. Cells that contain these expression cassettes should express high levels of the single chain antibodies in cells that are capable of activating the transgene promoter.

Production of transgenic mice is performed using standard techniques. Palmiter, RD, Brinster, RL. Ann. Rev. Genet. 20:465-499, 1988 Six injection days (or two weeks) are devoted to each construct. From this approach, approximately 30 mice are obtained. With a 10-30% transgenic rate, this should translate into anywhere from three to nine transgenic founder mice from each construct. Thus, three mice should be the minimum number of founders expected from any construct.

Transgenic founder mice and subsequent progeny are identified by the polymerase chain reaction using oligonucleotides complementary to the sequence encoding the antibody region of the heterologous nucleic acid. The founder transgenic animals are expected to exhibit specific and obvious phenotypes if CREB or ATF-2 are necessary for expression of either the GH or α-subunit genes. For example, in experiments involving overexpression of cholera toxin or a dominant negative CREB molecule, transgenic mice were either giant or dwarf, respectively. Burton, FH, Hasel, KW, Bloom, FE, Sutcliffe, JG. Nature 350:74-77, 1991. Thus, mice containing the GH promoter linked to the dominant negative antibodies should be dwarf while mice containing this promoter linked to the constitutively active antibodies would be expected to exhibit the giant phenotype.

In contrast to the phenotypes observed with GH promoter-directed antibodies, the α-antibody containing mice should be either hypo- or hypergonadal, depending on whether the dominant negative, or constitutively active, antibody is used. These phenotypes should be readily discernible by examination of external genitalia, gonadal histology, and reproductive capacity.

Lines of mice may be generated by breeding the founders to non-transgenic C57B/6 mice. Each male founder mouse is bred with three nontransgenic females while transgenic females are bred with a single nontransgenic male. Once F1 mice have been genotyped using the polymerase chain reaction (PCR), some will be assessed for antibody gene expression, while others are bred to perpetuate the lines.

Others have demonstrated for instance the efficient secretion of recombinant antibodies in a tissue-specific and developmentally regulated manner in the murine central nervous system. The local expression in the CNS of transgenic mice was used to perturb the function of the corresponding antigen. Piccioli, P., Di Luzio, A., Amann, R., Schuligoi, R., Surani, M.A., Donnerer, J., Neuron,15(2):373 (1995).

Expression of the sFv transgene in the appropriate cell types are confirmed using dual immunohistochemistry with antibodies directed against the fusion reagents and specific markers associated the cell such as LH (gonadotropes) or growth hormone (somatotrope), and in situ hybridization with probes directed to the coding sequences of the various proteins. Examination of serum levels of LH and GH as well as assessment of pituitary content of each of the hormones is also performed.

Pharmaceutical compositions

The present invention relates to pharmaceutical compositions which may comprise single chain monoclonal antibody fusion reagents alone or in combination with at least one other agent, such as stabilizing compound, which may be administered in nucleic acid form via gene therapy or in peptide form in any sterile, biocompatible pharmaceutical carrier, including, but not limited to, saline, buffered saline, dextrose, and water. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

Administration of pharmaceutical compositions

Administration of contemplated pharmaceutical compositions is accomplished orally or parenterally. Methods of parenteral delivery include topical, intra-arterial (directly to the affected tissue or tumor), intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers discussed comprising excipients and auxiliaries which facilitate processing of the active compounds into preparations which can be used pharmaceutically. Further details on techniques for formulation and administration may be found in the latest edition of “Remington's Pharmaceutical Sciences”(Maack Publishing Co, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, ie, dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the alt.

Manufacture and storage

The pharmaceutical compositions of the present invention may be manufactured in a manner that known in the art, eg, by means of conventional mixing, dissolving, granulating, dragee-making, levigating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a compound of the invention formulated in a acceptable carrier have been prepared, they can be placed in an appropriate container and labeled for treatment of an indicated condition.

Therapeutically effective dose

Pharmaceutical compositions suitable for use in the present invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, eg, of neoplastic cells, or in animal models including transgenic animals, usually mice, rabbits, dogs, or pigs. The animal model is also used to achieve a desirable concentration range and route of administration, Such information can then be used to determine useful doses and routes for administration in humans.

A therapeutically effective dose refers to that amount of single chain monoclonal antibody fusion reagent which ameliorate the symptoms or condition. Therapeutic efficacy and toxicity of such compounds can be determined by standard pharmaceutical procedures in cell cultures or experimental animals, eg, ED50 (the dose therapeutically effective in 50% of the population) and LD50 (the dose lethal to 50% of the population). The dose ratio between therapeutic and toxic effects is the therapeutic index, and it can be expressed as the ratio, ED50LD50. Pharmaceutical compositions which exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state, eg, tumor size and location; age, weight and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature. See U.S. Pat. Nos. 4,657,760; 5,206,344; or 5,225,212. Those skilled in the art will employ different formulations for nucleic acids than for peptide fusion reagents. Similarly, delivery of polynucleotides or polypeptides will be specific to particular cells, conditions or locations, for example.

It is contemplated that the single chain monoclonal antibody fusion reagents as well as nucleic acids discussed herein can be delivered in a suitable formulation to individuals having conditions where it is desirable to inhibit or enhance, respectively, the activity of genes and transcription associated biomolecules.

These examples are provided by way of illustration and are not included for the purpose of limiting the invention.

### EXAMPLES

I Construction of antigen bait strains

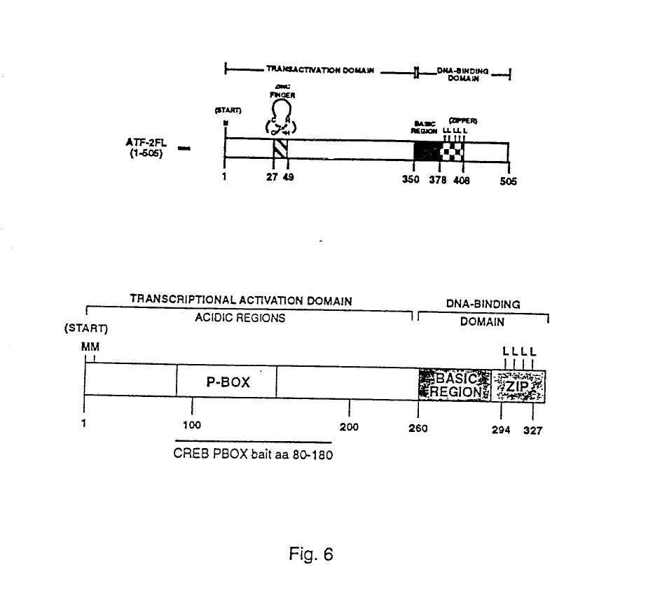

The first antigen bait chosen was the transcription factor ATF-2 (FIG.6A). Maekawa, T., et al., EMBO J., 8:2023 (1989).

The first bait construct was ATF-2FL cloned into Smal 5′ and BamH1 3′ of pBTM116 (FIG. 3). The yeast strain L40 (Mata his3 200 trp1-901 leu2-3,112 ade2 LYS::9lexAop)4-HIS3 URA3::(lexAop)8-LacZ Gal4) (Vojtek, AB, Hollenberg, SM, Cooper, JA. Cell 74:205-210, 1993) was tranformed with ATF-2FL/BTM116 using a frozen-EZ yeast transformation kit (ZYMO Research, Orange Calif.) and selected on plates lacking tryptophan. To confirm expression of the ATF-2FL/Lex A fusion protein, 5 OD units of the AFT-2FL/BTM116 transformed L40 were grown in selective media (YC-trp), pelleted, frozen on dry ice and the cell pellet was thawed in 100 μl cracking buffer (8M urea, 5% SDS, 40mM Tris-HCl ph 6.8. 0.1 mM EDTA, 1% β-mercaptoethanol, 0.4mg/ml bromophenol blue). The sample was then transferred to a 1.5 ml microfuge tube containing 100 μl glass beads, heated at 70° C. for 10 min, vortexed for 1 min and centrifuged for 5 min. 50 μl of the supernatant was separated on a 10% PAGE gel, transferred to nitrocellulose and probed with an anti-ATF-2FL polyclonal antibody raised in rabbits. Abdel-Hafiz, H., et al., Oncogene, 8:1161 (1993). The primary antibody was detected with HRP-conjugated donkey anti-rabbit IgG followed by ECL chemiluminescence. Lysate from the parent L40 strain was run as a negative control. Overexpression of the ATF-2FL/BTM116 was clearly shown in the bait strain.

The second antigen bait chosen was the transcription factor CREB (FIG. 6). Hoeffler, J. P., et al., Science, 242:1430 (1988). Amino acids 80-180 of CREB341 which contains the phosphorylation box were PCR amplified using standard conditions to incorporate a 5′ EcoR1 site and a 3′ Sall site for cloning into BTM116. Hoeffler, J. P., et al., Mol. Endocrinol., 4:920 (1990).

PCR Primers used:

| | | |
|---|---|---|
| Sense: | 5′ GTC GAA TTC CCA CAA GTC CAA ACA GTT CAG 3′ | (SEQ ID NO:89) |
| Antisense: | 5′ ACT GTC GAC TTA ATA CTG TCC ACT GCT AGT TTG 3′ | (SEQ ID NO:90) |

The CREBPBOX/BTM116 was transformed into L40 as described above and expression was verified by a western blot with an anti-CREBPBOX polyclonal antibody raised in rabbits. Ginty, D. D., et al., Science, 260:238 (1993).

II Library transformation

For the large-scale library transformations, we utilized a protocol that was a modification of the procedure described by Schiestl and Geitz. Scheistl, RH, Geitz, RD, Curr. Genet. 16:339-346, (1989).

1. Grow 5 ml overnight culture of the L40 bait strain in selective yeast media lacking trp and ura. Inoculate 100 ml of the same medium with an aliquot of the overnight culture. Grow overnight at 30° C. with constant shaking. The OD at 600 nm should be no greater than 4.0.

2. Add overnight culture from #1 above to a final OD₆₀₀ of 0.3 in 1 L YPAD (YEPD with 40 ug/ml adenine). Grow at 30° C. with constant shaking for 3 hours.

3. Pellet cells at room temperature by centrifugation at 2,500 rpm in a fixed angle rotor for a medium speed centrifuge. Decant supernatant.

4. Wash pellet in 500 ml of 1X TE.

5. Resuspend pellet in 20 ml 100 mM LiAc/0.5X TE and transfer to a sterlie 1 L flask.

6. Add DNA mixture: 1.0 ml of 10 mg/ml denatured salmon sperm DNA and 500 ug library DNA. Incubate with shaking for 16 hours.

7. Add 140 ml 100 mM LiAc/40% PEG-3350/1X TE. Mix, incubate 30 min at 30° C.

8. Add 17.6 ml DMSO. Swirl to mix. Heat shock at 42° C. for 6 min with occasional swirling to facilitate heat transfer. Imediately dilute with 400 ml of YPA and rapidly cool to room temperature in a water bath.

9. Pellet cells at 2,500 rpm for 5 minutes. Wash pellet with 500 mls YPA.

10. Resuspend pellet in 1L YPAD. Incubate at 30° C. for 1 hour with constant shaking.

11. Repeat steps 9 and 10. Plate 10 and 1 ul of the I L on selective yeast media lacking ura,trp, and leu. This measures primary transformation efficiency.

12. Repeat step 9 and wash pellet with selective media lacking trp, ura and leu and resuspend in 1L of this selective media.

13. Pellet cells and wash twice with selective media lacking trp, ura, leu and his and resuspend final pellet in 10 ml of this selective media.

14. Plate 10 plates each of 5 μl, 10 μl, 1, 25 μl and 50 μl on selective plates lacking trp, leu, ura and his. The His⁺ colonies that grow on selective plates represent colonies that were transforned with a library plasmidthat encodes an sFv fusion protein that recognizes the ATF-2/Lex A DBD or CREBPBOX fusion protein. To help rule out false positives the His+ colonies were duplicate plated onto plates lacking trp, ura, leu and his. After growth for 1 or 2 days, the colonies were analyzed for β-gal activity.

III Beta-galactosidase assays (filter method)

1 Lay a dry nitrocellulose filter onto the yeast colonies that are on selective media.

2. Remove the filter and float colony side up in a thin layer of liquid nitrogen. After 30 seconds, immerse filter for 5 seconds in the liquid nitrogen. Remove filter and place at room temperature, colony side up, until thawed.

3. Prepare a petri dish for the reaction. In the lid place 1.5 ml Z buffer containing 15 ul of 50 mg/ml X-gal (Z buffer=60 mM Na₂HPO₄, 40 mM Na₂ H₂PO₄, 10 mM KCl, 1 mM MgSO₄, pH 7.0). Lay 1# 1 Whatman filter circle in the Z buffer, followed by the nitrocellulose filter with colonies facing up. Cover with the bottom of the dish and place at 30° C. If longer incubations are required for positive signals to be visualized, the petri dish should be placed in a humidified chamber. Strong interactions yield detectable color in less than 30 min.

IV sFv library screens to isolate clones that target CREB and ATF-2

The first library screen was done with the ATF-2FL/BTM116 antigen bait strain. As shown in Flowchart I, approximately 4×10⁶ Trp⁺ Leu⁺ transformants were screened and 121 His⁺ clones were selected. Of the 121 His⁺ clones 72 were also β-Gal positive.

The sFv/VP16* plasmid DNA was isolated from several of the β-Gal positive clones and transferred to E. coli to facilitate further analysis. The procedure of Ward was used. Ward AC. Nucleic Acids Res. 18:5319, 1990. Basically, a 5 ml overnight culture is grown with the appropriate selection (media -trp, -leu, -his). The cells are pelleted, and the pellet is resuspended in 300 ul lysis buffer (2.5 M LiCl, 50 mM Tris-HCl (pH 8.0), 4% Triton X-100, 62.5 mM EDTA). The mixture is transferred to a 1.5 ml tube and 150 ul glass beads (0.45-0.50 mm) and 300 ul phenol/chloroform are added. This mixture is then vortexed vigorously for 1 minute. The beads and phenol/chloroform are pelleted in a microfuge for 1 minute, and the aqueous phase is transferred to a new tube. The plasinid DNA is precipitated twice with ethanol, and resuspended in 25 ul TE. 1-2 ul of this DNA is used to transform E. coli. After selection on LB plates with ampicillin the presence of the sFv/VP16* plasmid is screened for by restriction digest. The sFv clones.that were isolated and verified by restriction digest were further tested by transforming them back into yeast and analyzing there ablility to interact with different constructs. As shown in Flowchart I, transformation of the ATF-2FLsFvJVP16 clones alone or in combination with BTM116 or lamin/BTM116 gave a negative β-Gal reaction while transformation of the ATF-2FL/VP16 and the ATF-2FL/BTM116 did give a positive result as expected. These criteria represent verification of true postive clones islolated against the bait strain. Seven true positive ATF-2FLsFv/VP16 clones were isolated.

The second library screen was done against the CREBPBOX/BTM116 bait strain. In this screen approximately 5×10⁶ transfomants were screened and 185 His⁺ clones were selected. Of the 185 His⁺ clones selected 91 were also β-Gal positive. Twenty two CREBPBOXsFv/VP16 clones were isolated.

V Expression of sFv's in E. coli

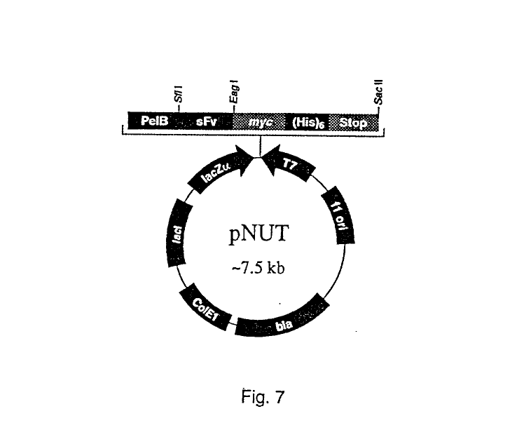

In order to evaluate the sFv clones isolated in the library screens, they were expressed in E. coli. The rationale behind this decision was to have a source of the sFv that we could use in vitro to test the sFv's abililty to recognize the bait protein on a western blot. As shown in FIG. 7 the expression plasmid pNUT was created with the Pel B leader upstream of the sFv. Enberg, J,. et al., Methods Mol. Biol., 51: 355 (1995); Better, M., et al., Science, 240:1041 (1988).

The sFv's were cloned into pNUT using SfiI 5′ and EagI 3′ to place the sFv in frame with a myc epitope tag (EQKLISEEDLN (SEQ ID NO: 91) which is recognized by the monoclonal antibody 9E10.2 (Evan, GI, Lewis, GK, Ramsay, G, Bishop, VM. Mol. Cell Biol. 5:3610-3616, 1985)) and His₆ for Ni purification. The sFv's could be easily shuttled into this vector from VP16* since they are cloned into VP16* SfiI and NotI. The sFv/VP16* clones were simply digested with SfiI and EagI (EagI is a 6 base cutter C′GGCCG that cuts within the 8 base NotI cutter GC′GGCCGC) and shuttled into SfiI/EagI cut pNUT. To verify the expression of the sFv's in pNUT the clones were transformed into HB101, grown to log phase in 2X YT with 0.1% glucose and 50μg/ml ampicillin and then induced with 0.1 mM IPTG overnight. Periplasmic preparations were then made by pelleting the bacteria and resuspending the pellet with osmotic lysis buffer (20% sucrose, 30 mM Tris pH 8.0, 1 mM EDTA, 1 mg/ml lysozyme) 1/40th volume (of original culture). The mixture was placed on ice for 10 min, centrifuged for 10 min at 10,000 rpm and the supernatant which contains the periplasmic preparation was saved. To verify that the sFv's were being expressed and were in frame with the myc epitope and the His₆ purification tag, 100 μl of the sFv periplasmic preparation was Ni purified with Probond resin (Invitrogen, San Diego, Calif.) and eluted with 500 mM imidazole. An anti-myc western blot of a CREBPBOXsFv/pNUT and a ATF2FLsFv/pNUT clone. Both sFv's are myc tagged and are able to be purified using the His₆ tag. All of the ATF-2FL and CREBPBOX sFv clones shuttled into pNUT to date produce sFv protein in this system. The sFv's were cloned into an expression vector to be epitope tagged because the sFv is comprised of only V_{H} and V_{L} chains and can therefore not be detected with a secondary antibody. Use of the epitope tagged sFv periplasmic preparation as a reagent to recognize the bait protein on a western blot requires the addition of anti-myc antibody also. The anti-myc antibody can then be detected with anti-mouse HRP followed by ECL chemiluminescence.

VI Characterization of targeting specificity of the isolated fusion reagent clones that target CREB and ATF-2 in vitro with bacterially expressed sFv's as reagents on western blots, and in vivo by expression of the fusion reagents in mammalian cells.

sUsing the fusion reagent periplasmic preparations in vitro

In order to test the ability of the ATF-2FL antibody fusion reagent to recognize its antigen bait (ATF2FL) in vitro a bacterial lysate of ATF-2FL/pRSET was used (pRSET: Invitrogen, San Diego, Calif.). An ATF-2FL/pRSET expressed protein was run on a 10% PAGE gel, transferred to nitrocellulose, probed with anti-ATF-2 polyclonal and detected with anti-rabbit HRP to verify presence of the ATF-2 FL protein.

This antibody fusion reagent preparation was capable of recognizing the bait antigen (ATF-2FL) that was screened in vivo in yeast. Four individual ATF-2FL antibody fusion periplasmic preparations were tested. Both ATF1 and ATF2 bacterial lysates were probed with the antibody fusions and showed that the ATF2 FL antibody fusions were specific and recognize ATF-2 but not ATF-1.

The same ATF-2 and ATF-1 bacterial lysates were also blotted and probed with anti-Xpress antibody (Invitrogen, San Diego, Calif.) which recognizes an epitope present in the pRSET vector that both the ATF-2 and ATF-1 clones were expressed in to show expression of both proteins.

The CREB antibody fusion clones ability to recognize its bait in vitro was also tested. The CREB antibody fusion clones did produce immunoglobulin protein which was demonstrated by a myc western of the periplasmic preparations.

VII Isolated clones that target CREB and ATF-2

In vivo targeting of the ATF-2sFv and CREBsFv antibody fusion reagents

In order to test the ability of the antibody fusion reagents to target their antigen baits in vivo both the ATF-2FL antibody/VP16 fusions and the CREB antibody/VP16 fusions were shuttled into pcDNA3.1 (Invitrogen, San Diego, Calif.). pcDNA3.1 is a eukaryotic expression plasmid which drives the gene of interest by the strong CMV promoter. This was done by digesting the original fusion clones isolated from the library screens with HindIII and EcoRI to isolate the antibody fusion reagent which contains the ATG, NLS, immunoglobulin domains, NLS, VP16, stop. These HindIII-EcoRI cassettes have been shuttled into HindIII/EcoRI digested pcDNA3. 1. These may be used for transfection into mammalian cells.

Stable cell lines may also be made, for instance in human JEG-3 choriocarcinoma cells and F9 embryonal carcinoma cells expressing a CRE-β-gal reporter construct to be used as a readout for the ability of the antibody fusion reagents to target and activate endogenous CREB or ATF-2. The CRE-β-gal reporter construct has an attenuated RSV promoter with 5 copies of the CRE element preceeding it. Pilz, RB, Suhasini, M, Idriss, S, Meinkoth, JK, Boss, GR. Faseb J. 9:552-558, 1995.

Two separate example model systems are contemplated to test the specificity and ability of the antibody fusion reagents to target endogenous DNA-bound CREB and ATF-2. A first model is the F9 embryonal carcinoma cell line. Endogenous levels of CREB and ATF-2 are extremely low, if at all detectable, in these cells, and they have been utilized extensively in the past for the investigation of individual CREB/ATF protein function. Meyer, TE, Habener, JF. Endocrine Reviews 14:269-290, 1993. The in vivo specificity in mammalian cells of the targeting by the antibody fusion reagents are expected to be further demonstrated utilizing this cell line.

A second cell model system contemplated is the human choriocarcinoma cell line JEG-3. These cells express the human alpha gonadotropin gene at high levels in response to increased intracellular levels of cyclic AMP (cAMP). The cells are known to contain “normal” endogenous levels of CREB and ATF-2. The antibody fusion reagents are expected to target endogenous DNA-bound transcription factors in this model system and activate the endogenous alpha-gonadotropin gene.

F9 Embrvonal Carcinoma Cells:

Since F9 cells have little or no endogenous CREB/ATF proteins, they are used as recipients for expression plasmids encoding either CREB and the CREB antibody fusion reagent, or ATF-2 and the ATF-2sFv antibody fusion reagent.

JEG-3 Human Choriocarcinoma Cells:

It is the goal of studies in this model system to target endogenous, DNA-bound transcriptional effectors with the antibody fusion reagents to influence gene expression from a promoter to which at least one of these factors is bound in vivo. The promoter of interest is the human alpha-gonadotropin gene promoter. It is known to contain two tandemly repeated copies of the CRE sequence and CREB from these cells will bind this sequence in the context of this promoter in in vitro assays of DNA-binding such as electrophoretic mobility shift assays (EMSA) and footprinting assays.

To test specificity in this assay system, a random antibody fusion reagent encoding sequence is amplified and cloned into pcDNA3.1 and subsequently transfected into the JEG-3 cells (as described supra for the CREB antibody fusion and ATF-2 fusion reagent encoding plasmids). This random antibody fusion reagent should have no effect on endogenous levels of the human alpha message. Therefore, the levels of human alpha message detected in cells transfected with this reagent are used as a point of comparison for levels of this message detected in cells transfected with the CREB antibody fusion or the ATF-2 targeting fusion reagents.

After the JEG-3 cell cultures are transfected with the plasmids expressing the random antibody fusion reagent, and the CREB and ATF-2 antibody fusion reagents by standard calcium phosphate precipitation techniques, the endogenous message for human alpha gonadotropin is measured at 6, 12, 24, and 48 hours post-transfection by standard Northern blot protocol. Northern results are nonnalized by probing the blots with a p-actin sequence and quantitation on a Molecular Dynamics Phosphor-imager.

VIII Construction of the yeast expression library vector pVP16Zeo

The yeast expression library vector pVP16Zeo (ATCC access #______) was constructed from three parent constructs: pPICZB (Invitrogen, San Diego), pGBT9 (Clonetech), and pVP16. Vojtek, A. B., Hollenberg, S. M., Cooper, J. A., Cell, 74:205 (1993). Selection in pVP16Zeo is based on a single selectable marker that confers resistance to the drug Zeocin in both Saccharoniyces cerevisiae and E. coli. Collis,CM, Hall, RM. Plasmid 14:143-151, 1985; Wenzel, TJ, Migliazza, A, Ydesteensma, H, Vandenberg JA. Yeast 8:667-668, 1992. Zeocin selection is also compatible with either trp or leu selectable markers which may be used as “bait” plasmid markers.

(1) The 1.9 kb Zeo fragment is obtained from pPICZB:

The parent construct is digested with Bgl II, and treated with T4 polymerase to form blunt ends. Bam HI digestion yields a blunt TEF1, EM7, Zeo^{r}, CYC1, E.coli ori., 1.9 kb Zeo fragment.

(2) The 0.9 kb promoter.Gal4 bd fragment, is obtained from pGBT9: The parent construct is digested with Sph I, and treated with T4 polymerase to form blunt ends. Bam HI digestion yields an ADH promoter, Gal 4 bd blunt 0.9 kb fagment.

(3) An intermediate 2.8 kb consruct is created by ligating the resulting fragments described in steps 1 and 2.

(4) The 2.3 kb VP16 . ADH terminator.2 μg origin fragment is obtained from pVP16: The parent construct is digested with Aat II, and treated with T4 polymerase to form blunt ends. Hind III digestion yields a blunt—nuclear localization signal, VP16 transactivation domain, ADH terminator, 2μ origin, 2.3 kb fragment. This 2.3 kb fragment is ligated into the intermediate 2.8 kb construct from step 3 (after the 2.8 kb construct from step 3 is digested with Hind III, Sma I to drop out a 0.4 kb Gal4 bd fragment) to yield pVP16Zeo.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. In deed, various modifications of the described modes for carrying out the invention which are obvious to those skilled in molecular biology or related fields are intended to be within the scope of the following claims.

What is claimed is:

1. A method of screening a DNA construct library for a single chain monoclonal antibody fusion reagent capable of binding a transcriptional associated biomolecule in vivo comprising:
cloning a nucleic acid fragment which encodes a peptide DBD of a transcription factor into an expression vector to yield a construct (1) such that the DBD may be expressed in a bio-active form and bind a corresponding DNA regulatory sequence binding site in a heterologous host cell,
fusing a nucleic acid fragment which encodes an antigenic portion of a transcriptional associated biomolecule into construct 1, in the same translation reading frame of the nucleic acid fragment which encodes the DBD of a transcription factor, to yield a construct (2),
cloning an sFv library into a DNA construct to yield a construct (3) such that a single chain monoclonal antibody may be expressed in bio-active form and bind a corresponding antigen in a heterologous host cell,
fusing a nucleic acid fragment which encodes a trans-activation peptide into construct 3, in the same translation reading frame of the nucleic acid fragment which encodes the single chain monoclonal antibody, to yield a construct (4) such that a resulting chimeric sFv/trans-activation peptide may be expressed in bio-active form and bind the corresponding antigen in a heterologous host cell,
providing a heterologous host cell harboring a detectable gene under transcriptional control of the DNA regulatory sequence binding site corresponding to the DBD encoded by construct 2,
introducing constructs 2 and 4 into the heterologous host cell harboring a detectable gene under transcriptional control of the DNA regulatory sequence binding site corresponding to the DBD encoded by construct 2, such that both constructs may be expressed, and
identifying a DNA construct 4 which encodes a single chain monoclonal antibody reagent capable of binding the transcriptional associated biomolecule in vivo by selecting for expression of the detectable gene.

2. The method of claim 1 further comprising fusing at least one nucleic acid fragment which encodes an intracellular targeting signal in the same translation reading frame to the nucleic acid fragment which encodes the single chain monoclonal antibody in construct 4, to yield a modified construct (5) such that a resulting single chain monoclonal antibody fusion reagent may be expressed in bio-active form and bind the corresponding antigen in a heterologous host cell.

3. The method of claim 1 further comprising fusing at least one nucleic acid fragment which encodes an intracellular targeting signal in the same translation reading frame to the nucleic acid fragment which encodes the single chain monoclonal antibody in construct 4, and deleting the TA, to yield a modified construct (6) such that a resulting single chain monoclonal antibody fusion reagent may be expressed in bio-active form and bind the corresponding antigen in a heterologous host cell.

4. The method of claim 2 wherein the transcriptional associated biomolecule is selected from the group consisting essentially of a transcription factor, ligand, honnone, nuclear hormone receptor, DNA binding domain of nuclear hormone receptor, tumor associated protein, protein kinase, protein phosphatase, GTP binding protein, adaptor protein, secondary messenger of an intracellular signaling molecule, and a protein derived from an etiological agent.

5. The method of claim 4 wherein the transcriptional associated biomolecule is selected from the group consisting of Ras, Grb2, phospholipase Cγ-PLCγ, phosphatidylinositol 3-kinase-PI3K, Syp, mitogen activated protein kinase-MAPK, jun kinase-JNK, androgen receptor (AR), thyroid hormone receptor (TR), glucocorticoid receptor (GR), ATF-1, ATF-2, ATF-3, ATF-4, ATF-6, CREB and CREMR.

6. The method of claim 3 wherein the transcriptional associated biomolecule is selected from the group consisting essentially of a transcription factor, ligand, hormone, nuclear hormone receptor, DNA binding domain of nuclear hormone receptor, tumor associated protein, protein kinase, protein phosphatase, GTP binding protein, adaptor protein, secondary messenger of an intracellular signaling molecule, and a protein derived from an etiological agent.

7. The method of claim 6 wherein the transcriptional associated biomolecule is selected from the group consisting of Ras, Grb2, phospholipase Cγ-PLCγ, phosphatidylinositol 3-kinase-PI3K, Syp, mitogen activated protein kinase-MAPK, jun kinase-JNK, androgen receptor (AR), thyroid hormone receptor (TR), glucocorticoid receptor (GR), ATF-1, ATF-2, ATF-3, ATF-4, ATF-6, CREB and CREMτ.

8. A method of screening a DNA construct library for a single chain monoclonal antibody fusion reagent capable of binding a transcriptional associated biomolecule in vivo comprising:
providing an expression construct (1) which encodes a peptide DBD of a transcription factor and comprises a cloning site for fusing a nucleic acid fragment which encodes an antigenic portion of a transcriptional associated biomolecule in the same translation reading frame of the nucleic acid fragment which encodes the DBD of a transcription factor, to yield a construct (2),
providing a DNA construct (3) which encodes a trans-activation peptide and comprises a cloning site for fusing an sFv library in the same translation reading frame of the trans-activation peptide, to yield a construct (4) such that a resulting chimeric sFv/trans-activation peptide may be expressed in bio-active form and bind a transcriptional associated biomolecule in a heterologous host cell,
providing a heterologous host cell, harboring a detectable gene under transcriptional control of the DNA regulatory sequence binding site corresponding to the DBD encoded by construct 2, for introducing constructs 2 and 4 into the heterologous host cell, such that both constructs may be expressed, and
identifying a DNA construct 4 which encodes a single chain monoclonal antibody reagent capable of binding the transcriptional associated biomolecule in vivo by selecting for expression of the detectable gene.

9. A single chain monoclonal antibody fusion reagent capable of binding a transcriptional associated biomolecule in vivo isolated by a method comprising:
cloning a nucleic acid fragment which encodes a peptide DBD of a transcription factor into an expression vector to yield a construct (1) such that the DBD may be expressed in a bio-active form and bind a corresponding DNA regulatory sequence binding site in a heterologous host cell,
fusing a nucleic acid fragment which encodes an antigenic portion of a transcriptional associated biomolecule into construct 1, in the same translation reading frame of the nucleic acid fragment which encodes the DBD of a transcription factor, to yield a construct (2),
cloning an sFv library into a DNA construct to yield a construct (3) such that a single chain monoclonal antibody may be expressed in bio-active form and bind a corresponding antigen in a heterologous host cell,
fusing a nucleic acid fragment which encodes a trans-activation peptide into construct 3, in the same translation reading frame of the nucleic acid fragment which encodes the single chain monoclonal antibody, to yield a construct (4) such that a resulting chimeric sFv/trans-activation peptide may be expressed in bio-active form and bind the corresponding antigen in a heterologous host cell,
providing a heterologous host cell harboring a detectable gene under transcriptional control of the DNA regulatory sequence binding site corresponding to the DBD encoded by construct 2,
introducing constructs 2 and 4 into the heterologous host cell harboring a detectable gene under transcriptional control of the DNA regulatory sequence binding site corresponding to the DBD encoded by construct 2, such that both constructs may be expressed,
identifying a DNA construct 4 which encodes a single chain monoclonal antibody reagent capable of binding the transcriptional associated biomolecule in vivo by selecting for expression of the detectable gene, and
isolating the single chain monoclonal antibody fusion reagent capable of binding the transcriptional associated biomolecule in vivo.

10. The single chain monoclonal antibody fusion reagent of claim 9 further comprising fusing at least one nucleic acid fragment which encodes an intracellular targeting signal in the same translation reading frame to the nucleic acid fragment which encodes the single chain monoclonal antibody in construct 4, to yield a modified construct (5) such that a resulting single chain monoclonal antibody fusion reagent may be expressed in bio-active form and bind the corresponding antigen in a heterologous host cell.

11. The single chain monoclonal antibody fusion reagent of claim 9 further comprising fusing at least one nucleic acid fragment which encodes an intracellular targeting signal in the same translation reading frame to the nucleic acid fragment which encodes the single chain monoclonal antibody in construct 4, and deleting the TA, to yield a modified construct (6) such that a resulting single chain monoclonal antibody fusion reagent may be expressed in bio-active form and bind the corresponding antigen in a heterologous host cell.

12. A single chain monoclonal antibody fusion reagent according to claim 9 which is capable of regulating transcription in vivo.

13. A single chain monoclonal antibody fusion reagent according to claim 10 which is capable of regulating transcription in vivo.

14. A single chain monoclonal antibody fusion reagent according to claim 11 which is capable of regulating transcription in vivo.

15. A therapeutic method for regulating the transcription of a gene in vivo comprising administering an effective amount of a single chain monoclonal antibody fusion reagent capable of binding a transcriptional associated biomolecule in vivo identified by a method comprising:
providing an expression construct (1) which encodes a peptide DBD of a transcription factor and comprises a cloning site for fusing a nucleic acid fragment which encodes an antigenic portion of a transcriptional associated biomolecule in the same translation reading frame of the nucleic acid fragment which encodes the DBD of a transcription factor, to yield a construct (2),
providing a DNA construct (3) which encodes a trans-activation peptide and comprises a cloning site for fusing an sFv library in the same translation reading frame of the trans-activation peptide, to yield a construct (4) such that a resulting chimeric sFv/trans-activation peptide may be expressed in bio-active form and bind a transcriptional associated biomolecule in a heterologous host cell,
providing a heterologous host cell, harboring a detectable gene under transcriptional control of the DNA regulatory sequence binding site corresponding to the DBD encoded by construct 2, for introducing constructs 2 and 4 into the heterologous host cell, such that both constructs may be expressed, and
identifying a DNA construct 4 which encodes a single chain monoclonal antibody reagent capable of binding the transcriptional associated biomolecule in vivo by selecting for expression of the detectable gene.

16. A therapeutic method for regulating the transcription of a gene in vivo according to claim 15 wherein the single chain monoclonal antibody fusion reagent capable of binding a transcriptional associated biomolecule in vivo comprises at least one intracellular targeting signal fused to the single chain monoclonal antibody.

17. A method of screening a plurality of compounds for specific binding affinity with a single chain monoclonal antibody fusion reagent capable of binding a transcriptional associated biomolecule in vivo identified by a method comprising:
providing an expression construct (1) which encodes a peptide DBD of a transcription factor and comprises a cloning site for fusing a nucleic acid fragment which encodes an antigenic portion of a transcriptional associated biomolecule in the same translation reading frame of the nucleic acid fragment which encodes the DBD of a transcription factor, to yield a construct (2),
providing a DNA construct (3) which encodes a trans-activation peptide and comprises a cloning site for fusing an sFv library in the same translation reading frame of the trans-activation peptide, to yield a construct (4) such that a resulting chimeric sFv/trans-activation peptide may be expressed in bio-active form and bind a transcriptional associated biomolecule in a heterologous host cell,
providing a heterologous host cell, harboring a detectable gene under transcriptional control of the DNA regulatory sequence binding site corresponding to the DBD encoded by construct 2, for introducing constructs 2 and 4 into the heterologous host cell, such that both constructs may be expressed, and
identifying a DNA construct 4 which encodes a single chain monoclonal antibody reagent capable of binding the transcriptional associated biomolecule in vivo by selecting for expression of the detectable gene, and
screening a plurality of compounds comprising the steps of:
providing a plurality of compounds, combining the single chain monoclonal antibody fusion reagent with each of a plurality of compounds for a time sufficient to allow binding under suitable conditions; and
detecting binding of said single chain monoclonal antibody fusion reagent to each of the plurality of compounds, thereby identifying the compounds which specifically bind said single chain monoclonal antibody fusion reagent.

18. A method for diagnosing a physiological disorder manifested by abnormal levels of a transcription associated biomolecule, said method comprising:
contacting a biological sample with a labelled single chain monoclonal antibody fusion reagent or a portion thereof according to claim 9 whereby said antibody reagent binds to said transcription associated biomolecule to form a complex,
separating unbound labelled antibody reagent from said complex,
measuring the amount of bound labelled antibody reagent in said complex; and,
comparing the quantity of labelled antibody reagent in said biological sample to the quantity of labelled antibody reagent which binds to normal biological samples under identical conditions.

19. A pVP16Zeo library expression vector (ATCC deposit #______) for the construction and screening of single chain monoclonal antibody fusion reagent libraries, comprising zeocin selection to facilitate the isolation and production of single chain monoclonal antibody fusion reagents in yeast and E. coli.

20. A kit for screening a DNA construct library for a single chain monoclonal antibody fusion reagent capable of binding a transcriptional associated biomolecule in vivo; comprising in a container:
an expression construct (1) which encodes a peptide DBD of a transcription factor and comprises a cloning site for fusing a nucleic acid fragment which encodes an antigenic portion of a transcriptional associated biomolecule in the same translation reading frame of the nucleic acid fragment which encodes the DBD of a transcription factor, to yield a construct (2), and
a DNA construct (3) which encodes a trans-activation peptide and comprises a cloning site for fusing an sFv library in the same translation reading frame of the trans-activation peptide, to yield a construct (4) such that a resulting chimeric sFv/trans-activation peptide may be expressed in bio-active form and bind a transcriptional associated biomolecule in a heterologous host cell, and
a heterologous host cell harboring a detectable gene under transcriptional control of the DNA regulatory sequence binding site corresponding to the DBD encoded by construct 2, for introducing constructs 2 and 4 into the heterologous host cell, such that both constructs may be expressed, and
a means for identifying a DNA construct 4 which encodes a single chain monoclonal antibody reagent capable of binding the transcriptional associated biomolecule in vivo by selecting for expression of the detectable gene.

21. A kit for screening a DNA construct library for a single chain monoclonal an tibody fusion reagent capable of binding a transcriptional associated biomolecule in vivo according to claim 20 wherein DNA construct 3 is pVP16Zeo (ATCC deposit #______).

22. A kit for screening a DNA construct library for a single chain monoclonal antibody fusion reagent capable of binding a transcriptional associated biomolecule in vivo according to claim 21 wherein primers are provided for human sFv library construction.

23. A kit for screening a DNA construct library for a single chain monoclonal antibody fusion reagent capable of binding a transcriptional associated biomolecule in vivo according to claim 22 wherein primers select e d from the group consisting essentially of (SEQ ID NOs: 3-8. are provided for human sFv library construction.


SEQUENCE LISTING (96 sequences)

SEQ ID NO: 1 - protein, 29 aa, Artificial Sequence
  Description of Artificial Sequence Illustrative mitochondrial target signal
MSVLTPLLLRGLTGSARRLPVPRAKIHSL

SEQ ID NO: 2 - protein, 20 aa, Artificial Sequence
  Description of Artificial Sequence Illustrative mitochondrial target signal
METDLLLWVLLLWVPGSTGD

SEQ ID NO: 3 - DNA, 24 bp, Artificial Sequence
  Description of Artificial Sequence Primer
tggaagaggcacgttcttttcttt

SEQ ID NO: 4 - DNA, 24 bp, Artificial Sequence
  Description of Artificial Sequence Primer
gtccaccttggtgttgctgggctt

SEQ ID NO: 5 - DNA, 24 bp, Artificial Sequence
  Description of Artificial Sequence Primer
agactctcccctgttgaagctctt

SEQ ID NO: 6 - DNA, 27 bp, Artificial Sequence
  Description of Artificial Sequence Primer
tgaagattctgtaggggccactgtctt

SEQ ID NO: 7 - DNA, 23 bp, Artificial Sequence
  Description of Artificial Sequence Primer
caggtgcagctggtgcagtctgg

SEQ ID NO: 8 - DNA, 23 bp, Artificial Sequence
  Description of Artificial Sequence Primer
caggtgcagctggtggagtctgg

SEQ ID NO: 9 - DNA, 23 bp, Artificial Sequence
  Description of Artificial Sequence Primer
caggtccagcttgtgcagtctgg

SEQ ID NO: 10 - DNA, 23 bp, Artificial Sequence
  Description of Artificial Sequence Primer
caggtcaccttgaaggagtctgg

SEQ ID NO: 11 - DNA, 23 bp, Artificial Sequence
  Description of Artificial Sequence Primer
cagatcaccttgaaggagtctgg

SEQ ID NO: 12 - DNA, 23 bp, Artificial Sequence
  Description of Artificial Sequence Primer
caggtgcagctggtggagtctgg

SEQ ID NO: 13 - DNA, 23 bp, Artificial Sequence
  Description of Artificial Sequence Primer
caggtgcagctgttgcagtctgg

SEQ ID NO: 14 - DNA, 23 bp, Artificial Sequence
  Description of Artificial Sequence Primer
caggtgcagctgttgcagtctgc

SEQ ID NO: 15 - DNA, 23 bp, Artificial Sequence
  Description of Artificial Sequence Primer
gaggtgcagctggtgcagtctgg

SEQ ID NO: 16 - DNA, 23 bp, Artificial Sequence
  Description of Artificial Sequence Primer
gaggtgcagctgttggagtctgg

SEQ ID NO: 17 - DNA, 23 bp, Artificial Sequence
  Description of Artificial Sequence Primer
caggtacagctgcagcagtcagg

SEQ ID NO: 18 - DNA, 54 bp, Artificial Sequence
  Description of Artificial Sequence Primer
gtcctcgcaactgcggcccagccggccatggcccaggtgcagctggtgcagtct

SEQ ID NO: 19 - DNA, 56 bp, Artificial Sequence
  Description of Artificial Sequence Primer
gtcctcgcaactgcggcccagccggccatggcccaggtgcagctggtggagtctgg

SEQ ID NO: 20 - DNA, 56 bp, Artificial Sequence
  Description of Artificial Sequence Primer
gtcctcgcaactgcggcccagccggccatggcccaggtccagcttgtgcagtctgg

SEQ ID NO: 21 - DNA, 56 bp, Artificial Sequence
  Description of Artificial Sequence Primer
gtcctcgcaactgcggcccagccggccatggcccaggtcaccttgaaggagtctgg

SEQ ID NO: 22 - DNA, 56 bp, Artificial Sequence
  Description of Artificial Sequence Primer
gtcctcgcaactgcggcccagccggccatggcccagatcaccttgaaggagtctgg

SEQ ID NO: 23 - DNA, 56 bp, Artificial Sequence
  Description of Artificial Sequence Primer
gtcctcgcaactgcggcccagccggccatggccgaggtgcagctggtggagtctgg

SEQ ID NO: 24 - DNA, 56 bp, Artificial Sequence
  Description of Artificial Sequence Primer
gtcctcgcaactgcggcccagccggccatggcccaggtgcagctgcaggagtcggg

SEQ ID NO: 25 - DNA, 56 bp, Artificial Sequence
  Description of Artificial Sequence Primer
gtcctcgcaactgcggcccagccggccatggccgaggtgcagctgttgcagtctgc

SEQ ID NO: 26 - DNA, 56 bp, Artificial Sequence
  Description of Artificial Sequence Primer
gtcctcgcaactgcggcccagccggccatggccgaggtgcagctggtgcagtctgg

SEQ ID NO: 27 - DNA, 56 bp, Artificial Sequence
  Description of Artificial Sequence Primer
gtcctcgcaactgcggcccagccggccatggccgaggtgcagctgttggagtctgg

SEQ ID NO: 28 - DNA, 56 bp, Artificial Sequence
  Description of Artificial Sequence Primer
gtcctcgcaactgcggcccagccggccatggcccaggtccagctgcagcagtcagg

SEQ ID NO: 29 - DNA, 24 bp, Artificial Sequence
  Description of Artificial Sequence Primer
tgaggagacggtgaccagggtgcc

SEQ ID NO: 30 - DNA, 24 bp, Artificial Sequence
  Description of Artificial Sequence Primer
tgaggagacagtgaccagggtgcc

SEQ ID NO: 31 - DNA, 24 bp, Artificial Sequence
  Description of Artificial Sequence Primer
tgaagagacggtgaccattgtccc

SEQ ID NO: 32 - DNA, 24 bp, Artificial Sequence
  Description of Artificial Sequence Primer
tgaggagacggtgaccagggtccc

SEQ ID NO: 33 - DNA, 24 bp, Artificial Sequence
  Description of Artificial Sequence Primer
tgaggagacggtgaccagggttcc

SEQ ID NO: 34 - DNA, 24 bp, Artificial Sequence
  Description of Artificial Sequence Primer
tgaggagacggtgaccgtggtccc

SEQ ID NO: 35 - DNA, 36 bp, Artificial Sequence
  Description of Artificial Sequence Primer
gagtcattctcgtgtcgtcacggtgaccagggtgcc

SEQ ID NO: 36 - DNA, 36 bp, Artificial Sequence
  Description of Artificial Sequence Primer
gagtcattctcgtgtcgacacagtgaccagggtgcc

SEQ ID NO: 37 - DNA, 36 bp, Artificial Sequence
  Description of Artificial Sequence Primer
gagtcattctcgtgtcgacacggtgaccattgtccc

SEQ ID NO: 38 - DNA, 36 bp, Artificial Sequence
  Description of Artificial Sequence Primer
gagtcattctcgtgtcgacacggtgaccagggtccc

SEQ ID NO: 39 - DNA, 36 bp, Artificial Sequence
  Description of Artificial Sequence Primer
gagtcattctcgtgtcgacacggtgaccagggttcc

SEQ ID NO: 40 - DNA, 36 bp, Artificial Sequence
  Description of Artificial Sequence Primer
gagtcattctcgtgtcgacacggtgaccgtggtccc

SEQ ID NO: 41 - DNA, 23 bp, Artificial Sequence
  Description of Artificial Sequence Primer
gacatccagatgacccagtctcc

SEQ ID NO: 42 - DNA, 23 bp, Artificial Sequence
  Description of Artificial Sequence Primer
gatattgtgatgacccagwctcc

SEQ ID NO: 43 - DNA, 23 bp, Artificial Sequence
  Description of Artificial Sequence Primer
gaaattgtgytgacwcagtctcc

SEQ ID NO: 44 - DNA, 23 bp, Artificial Sequence
  Description of Artificial Sequence Primer
gacatcgtgatgacccagtctcc

SEQ ID NO: 45 - DNA, 23 bp, Artificial Sequence
  Description of Artificial Sequence Primer
gaaacgacactcacgcagtctcc

SEQ ID NO: 46 - DNA, 23 bp, Artificial Sequence
  Description of Artificial Sequence Primer
gagattgtgatgacccagactcc

SEQ ID NO: 47 - DNA, 23 bp, Artificial Sequence
  Description of Artificial Sequence Primer
gaccacgtgatgacccagtctcc

SEQ ID NO: 48 - DNA, 41 bp, Artificial Sequence
  Description of Artificial Sequence Primer
tgagcacacagtgcactcgacatccagatgacccagtctcc

SEQ ID NO: 49 - DNA, 41 bp, Artificial Sequence
  Description of Artificial Sequence Primer
tgagcacacagtgcactcgatattgtgatgacccagwctcc

SEQ ID NO: 50 - DNA, 41 bp, Artificial Sequence
  Description of Artificial Sequence Primer
tgagcacacagtgcactcgaaattgtgytgacwcagtctcc

SEQ ID NO: 51 - DNA, 41 bp, Artificial Sequence
  Description of Artificial Sequence Primer
tgagcacacagtgcactcgacatcgtgatgacccagtctcc

SEQ ID NO: 52 - DNA, 41 bp, Artificial Sequence
  Description of Artificial Sequence Primer
tgagcacacagtgcactcgaaacgacactcacgcagtctcc

SEQ ID NO: 53 - DNA, 41 bp, Artificial Sequence
  Description of Artificial Sequence Primer
tgagcacacagtgcactcgagattgtgatgacccagactcc

SEQ ID NO: 54 - DNA, 41 bp, Artificial Sequence
  Description of Artificial Sequence Primer
tgagcacacagtgcactcgaccacgtgatgacccagtctcc

SEQ ID NO: 55 - DNA, 24 bp, Artificial Sequence
  Description of Artificial Sequence Primer
acgtttgatytccascttggtccc

SEQ ID NO: 56 - DNA, 24 bp, Artificial Sequence
  Description of Artificial Sequence Primer
acgtttgatatccactttggtccc

SEQ ID NO: 57 - DNA, 24 bp, Artificial Sequence
  Description of Artificial Sequence Primer
acgtttaatctccagtcgtgtccc

SEQ ID NO: 58 - DNA, 48 bp, Artificial Sequence
  Description of Artificial Sequence Primer
gagtcattctcgacttgcggccgcacgtttgatytccascttggtccc

SEQ ID NO: 59 - DNA, 48 bp, Artificial Sequence
  Description of Artificial Sequence Primer
gagtcattctcgacttgcggccgcacgtttgatatccactttggtccc

SEQ ID NO: 60 - DNA, 48 bp, Artificial Sequence
  Description of Artificial Sequence Primer
gagtcattctcgacttgcggccgcacgtttaatctccagtcgtgtccc

SEQ ID NO: 61 - DNA, 23 bp, Artificial Sequence
  Description of Artificial Sequence Primer
cagtctgtgytgackcagccgcc

SEQ ID NO: 62 - DNA, 23 bp, Artificial Sequence
  Description of Artificial Sequence Primer
ctgtgctgackcagccrccctca

SEQ ID NO: 63 - DNA, 23 bp, Artificial Sequence
  Description of Artificial Sequence Primer
cagtctgccctgactcagcctgc

SEQ ID NO: 64 - DNA, 23 bp, Artificial Sequence
  Description of Artificial Sequence Primer
cagactgtggtgacccaggagcc

SEQ ID NO: 65 - DNA, 23 bp, Artificial Sequence
  Description of Artificial Sequence Primer
tcctctgagctgagtcagcagcc

SEQ ID NO: 66 - DNA, 23 bp, Artificial Sequence
  Description of Artificial Sequence Primer
caggctgtggtgactcaggagcc

SEQ ID NO: 67 - DNA, 23 bp, Artificial Sequence
  Description of Artificial Sequence Primer
ctgtggtgacccaggagccatca

SEQ ID NO: 68 - DNA, 23 bp, Artificial Sequence
  Description of Artificial Sequence Primer
cagcctgtgctgactcagccacc

SEQ ID NO: 69 - DNA, 23 bp, Artificial Sequence
  Description of Artificial Sequence Primer
cctatgagctgactcagccactc

SEQ ID NO: 70 - DNA, 41 bp, Artificial Sequence
  Description of Artificial Sequence Primer
tgagcacacagtgcactccagtctgtgytgackcagccgcc

SEQ ID NO: 71 - DNA, 41 bp, Artificial Sequence
  Description of Artificial Sequence Primer
tgagcacacagtgcactcctgtgctgackcagccrccctca

SEQ ID NO: 72 - DNA, 41 bp, Artificial Sequence
  Description of Artificial Sequence Primer
tgagcacacagtgcactccagtctgccctgactcagcctgc

SEQ ID NO: 73 - DNA, 41 bp, Artificial Sequence
  Description of Artificial Sequence Primer
tgagcacacagtgcactccagactgtggtgacccaggagcc

SEQ ID NO: 74 - DNA, 41 bp, Artificial Sequence
  Description of Artificial Sequence Primer
tgagcacacagtgcactctcctctgagctgagtcagcagcc

SEQ ID NO: 75 - DNA, 41 bp, Artificial Sequence
  Description of Artificial Sequence Primer
tgagcacacagtgcactccaggctgtggtgactcaggagcc

SEQ ID NO: 76 - DNA, 41 bp, Artificial Sequence
  Description of Artificial Sequence Primer
tgagcacacagtgcactcctgtggtgacccaggagccatga

SEQ ID NO: 77 - DNA, 41 bp, Artificial Sequence
  Description of Artificial Sequence Primer
tgagcacacagtgcactccagcctgtgctgactcagccacc

SEQ ID NO: 78 - DNA, 41 bp, Artificial Sequence
  Description of Artificial Sequence Primer
tgagcacacagtgcactccctatgagctgactcagccactc

SEQ ID NO: 79 - DNA, 24 bp, Artificial Sequence
  Description of Artificial Sequence Primer
acctaggacggtgaccttggtccc

SEQ ID NO: 80 - DNA, 24 bp, Artificial Sequence
  Description of Artificial Sequence Primer
acctaggacggtcagctyggtccc

SEQ ID NO: 81 - DNA, 24 bp, Artificial Sequence
  Description of Artificial Sequence Primer
acctaaaatgatcagctgggttcc

SEQ ID NO: 82 - DNA, 24 bp, Artificial Sequence
  Description of Artificial Sequence Primer
accgaggacggtcaccttggtggc

SEQ ID NO: 83 - DNA, 42 bp, Artificial Sequence
  Description of Artificial Sequence Primer
gagtcattctcgacttgcggccgcacctaggacctyggtccc

SEQ ID NO: 84 - DNA, 48 bp, Artificial Sequence
  Description of Artificial Sequence Primer
gagtcattctcgacttgcggccgcacctaggacggtcagcttggtccc

SEQ ID NO: 85 - DNA, 48 bp, Artificial Sequence
  Description of Artificial Sequence Primer
gagtcattctcgacttgcggccgcacctaaaatgatcagctgggttcc

SEQ ID NO: 86 - DNA, 48 bp, Artificial Sequence
  Description of Artificial Sequence Primer
gagtcattctcgacttgcggccgcaccgaggacggtcaggttggtggc

SEQ ID NO: 87 - DNA, 38 bp, Artificial Sequence
  Description of Artificial Sequence Primer
agtggcccagccggccaaattcaagttacatgtgaatt

SEQ ID NO: 88 - DNA, 34 bp, Artificial Sequence
  Description of Artificial Sequence Primer
gaggcggccgcacttcctgagggctgtgactggg

SEQ ID NO: 89 - DNA, 30 bp, Artificial Sequence
  Description of Artificial Sequence Primer
gtcgaattcccacaagtccaaacagttcag

SEQ ID NO: 90 - DNA, 33 bp, Artificial Sequence
  Description of Artificial Sequence Primer
actgtcgacttaatactgtccactgctagtttg

SEQ ID NO: 91 - protein, 11 aa, Artificial Sequence
  Description of Artificial Sequence myc epitope tag
EQKLISEEDLN

SEQ ID NO: 92 - protein, 8 aa, Artificial Sequence
  Description of Artificial Sequence Synthetic peptide
DPKKKRKV

SEQ ID NO: 93 - protein, 6 aa, Artificial Sequence
  Description of Artificial Sequence Illustrative endoplasmic reticulum retention signal
SEKDEL

SEQ ID NO: 94 - protein, 6 aa, Artificial Sequence
  Description of Artificial Sequence 6-His tag
HHHHHH

SEQ ID NO: 95 - DNA, 28 bp, Artificial Sequence
  Description of Artificial Sequence Synthetic oligonucleotide
gaattcccggggatccgtcgacctgcag

SEQ ID NO: 96 - protein, 15 aa, Artificial Sequence
  Description of Artificial Sequence Peptide linker
GGGGSGGGGSGGGGS